(19) United States
(12) Patent Application Publication
(10) Pub. No.: US 2025/0271896 A1
(43) Pub. Date: Aug. 28, 2025

(54) FOLDABLE SENSOR-BASED DEVICES

(71) Applicants: Nelson L. JUMBE, Mountain View, CA (US); Andreas SCHUH, Millbrae, CA (US); Peter REXELIUS, San Francisco, CA (US); Michael MORIMOTO, Dublin, CA (US); Steve RODRIGUEZ, Mountain View, CA (US); Mark HAUN, Mountain View, CA (US); Chris ZHANG, Mountain View, CA (US)

(72) Inventors: Nelson L. JUMBE, Mountain View, CA (US); Andreas SCHUH, Millbrae, CA (US); Peter REXELIUS, San Francisco, CA (US); Michael MORIMOTO, Dublin, CA (US); Steve RODRIGUEZ, Mountain View, CA (US); Mark HAUN, Mountain View, CA (US); Chris ZHANG, Mountain View, CA (US)

(21) Appl. No.: 18/256,990

(22) PCT Filed: Dec. 13, 2021
(86) PCT No.: PCT/US2021/063151

§ 371 Date: Jun. 12, 2023

Related U.S. Application Data
(60) Provisional application No. 63/124,593, filed on Dec. 11, 2020.

(51) Int. Cl. G06F 1/16; G06F 1/18; G06F 3/01; G06F 3/05

(52) U.S. Cl. CPC G06F 1/163; G06F 1/1635; G06F 1/189; G06F 3/015; G06F 3/05

(57) ABSTRACT

There is disclosed a foldable sensor device. The foldable sensor device comprises a first substrate, a second substrate, and a join member. The first substrate has at least one sensor. The second substrate has at least one sensor. The join member connects the first substrate and the second substrate. The first substrate and the second substrate are foldable relative to each other to form a stacked configuration where the first substrate is stacked relative to the second substrate.

### CROSS-REFERENCE TO RELATED APPLICATION

This application is a National Phase Entry of International Patent application no. PCT/US2021/063151 filed on Dec. 13, 2021 and which claims the benefit of U.S. Provisional Patent Application No. 63/124,593, filed on Dec. 11, 2020, the contents of the above noted application are incorporated by reference herein in their entirety.

### TECHNICAL FIELD

This disclosure generally relates to sensor-based devices, such as but not limited to sensor-based devices that can measure signals associated with a user of the sensor-based device.

### BACKGROUND

Sensor devices exist for a multitude of uses, such as in the form of wearable devices for detecting physiological signals from a subject, or free-standing devices for measuring ambient conditions.

For certain uses, it may be desirable to combine sensors for detecting both physiological signals from the user as well as measuring ambient conditions in an environment of the user in a single device.

However, it will be appreciated that combining such sensor-types into a single device is not readily apparent. Proximity and a relative angle of sensors to one another, as well as to the target object from which the signals are to be detected, can have detrimental impact on the signal-to-noise ratio of the signals being detected

It is therefore desired to provide a sensor-based device which overcomes or reduces the above-noted shortcomings,

### SUMMARY

The present technology relates to the development of sensor-based devices which address or overcome the above-noted shortcomings.

From a broad aspect, there are provided sensor-based devices having a foldable configuration for forming a multi-layered structure from a planar structure. In certain embodiments, the sensor-based devices include a plurality of substrates for housing a plurality of sensors, the plurality of substrates being substantially co-planar in an unfolded configuration and stacked in a folded configuration. In the stacked configuration, the substrates may be substantially parallel or orthogonal to one another thereby defining a multi-layered structure. There are a number of advantages with such a sensor-based device, not least relating to manufacturing ease and improved functionality. With regard to manufacturing ease, sensors and other electronic components can be connected together on a planar configuration. Subsequent folding can create a stacked multi-layered configuration which has a smaller footprint than the unfolded configuration. Smaller footprints are advantageous for many uses, and particularly for wearable applications in which discreteness is preferred. Furthermore, such a multi-layered sensor device can be useful for positioning sensors on different planes thereof and therefore at different proximities to a target. Additionally, sensors can be pointed in different directions. For example, sensors for detecting signals associated with a subject wearing the device may be pointed towards the subject and closer to the subject and sensors for detecting environmental parameters may be pointed towards the environment (away from the subject) and further from the subject.

Furthermore, certain aspects and embodiments of the present technology may include for example multi-dimension data stream fusion, with edge and cloud-based domain adaptable recursive stochastic synthesis programming. Certain embodiments of the present technology also provide for the use of said sensor devices with communications networks and computing platforms, to create models, detect changes, and perform detailed condition-based monitoring and health failure prediction.

By monitoring signal data relating to the subject and the environment, correlations can be made between a condition of the subject and the environment, and such correlations can be used to enhance a current condition of the subject (whether living or inanimate), maintain a current condition of the subject, prevent a certain condition (such as disease states) of the subject, and/or predict certain conditions of the subject. For example, such data may allow anomalous health incidents (AHI) to be identified. One example of an AHI is due to directed energy weapons such as those associated with the Havana Syndrome. Directed energy weapons may include beams of energies over a broad spectrum of infrasound, radio, sonic and microwave frequencies that cause a range of temporary or permanent health effects which in certain cases resemble decompression sickness. In certain embodiments, the present technology can therefore establish a baseline for a subject by monitoring physiological parameters such as respiration, blood pressure, electrocardiogram signal, sleep analysis. The baseline may be a dynamic baseline, i.e. updated. An AHI may be identified as anomalous perturbations to the baseline.

Therefore, the present technology aims to provide solutions towards health state/condition, health status condition monitoring, symptom screening and diagnostics, as well as overall physical performance enhancement in living bodies. This differs from traditional approaches of current symptom and disease management.

This objective is also important in health monitoring of inanimate structures, such as buildings, dams, bridges, as well as in the efficient use of animate structures such as rotating and reciprocating machinery, vibrating structures, mechanical facilities and technical systems in the industrial and construction sector, and wherever else machines are used. Humans, rotating structures and machines have traditionally been placed on an arbitrary service plan; milestone-driven vaccine and/or yearly physical physician office visits, or mileage/rotation triggered maintenance, where late maintenance means destructive wear and a risk of production downtime.

According to certain aspects, there are provided devices, methods and systems for condition-based monitoring and/or health failure prediction in animals, plants, as well as non-living structures.

From a broad aspect, the devices of the present technology comprise multiple layers for supporting a plurality of sensors. Supporting sensors on different layers of such a device can provide a more efficient monitoring of both a subject which is wearing the device as well as the environment around the subject. Given sensors can thereby be positioned closer to the object that they are monitoring for more efficient signal pick-up. This can be advantageous for further processing of the signals detected in terms of signal-to-noise ratios.

Advantageously, in certain aspects and embodiments, such multi-layer devices may be formed by a folding mechanism in which the different layers are provided initially on a single plane and then folded relative to each other to form the multi-layer device. This can provide a multitude of savings in terms of manufacturing time and costs. In this respect, embodiments of the present technology extend to both an unfolded (i.e. “flat”) configuration of the device of the present technology as well as a folded (or “stacked”) configuration.

In certain aspects, there are also provided methods of monitoring a health condition of a body using such devices. In this respect, Developers of the present technology have also developed methods for prolonging a life data capture time of the sensors of the device by applying a data capture protocol to a battery associated with the sensors.

From one aspect, there is provided a foldable sensor device comprising: a first substrate having a first sensor; a second substrate having a second sensor; and a first join member connecting the first substrate and the second substrate such that the first substrate and the second substrate are foldable relative to each other to form a folded configuration having multiple layers with the first substrate stacked relative to the second substrate. The foldable sensor device may include any number of substrates, such as 2, 3, 4, 5, 6, 7, 8, 9, or 10.

In certain embodiments, the foldable sensor device further comprises an enclosure housing the first substrate, the second substrate and the first join member when the first substrate and the second substrate are in the folded configuration.

In certain embodiments, the foldable sensor device further comprises a retaining member for retaining the first substrate and the second substrate in the folded configuration. The retaining member may be a clip or any other component that can maintain a distance of the outermost substrates to each other.

In certain embodiments, the first sensor is configured to detect signals from a user of the foldable sensor device and the second sensor is configured to detect signals from an environment of the user, and wherein the first sensor faces away from the second sensor.

In certain embodiments, the first sensor is positioned on a first surface of the first substrate and the second surface is positioned on a second surface of the second substrate, the first surface and the second surface being co-planar when in an unfolded configuration and stacked one above each other when in a folded configuration. The first surface may face away from the second surface, or in cases in which the join member includes a twist, the first and second surfaces may face in the same direction.

In certain embodiments, at least a portion of one or both of the first sensor and the second sensor are formed within a first body of the first substrate or a second body of the second substrate. For example, a portion of the first substrate or the second substrate may comprise a filter.

In certain embodiments, the join member comprises one or more of a flexible material, a hinge or an accordion-like structure. The flexible material may comprise a flexible PCB material such as one or more of polyimide film, a polyester film, polyethylene nphthalate, polytetrafluoroethylene, aramid, and copper foil.

In certain embodiments, the first substrate and/or the second substrate may be one-piece with the first join member.

In certain embodiments, the enclosure has a configuration which is wearable by a user against or proximate a body part of the user and which is selected from one or more of: a strap, a band aid, a patch, a watch, a bandage, an item of jewelry, a head piece, an eye piece, an ear piece, a mouth piece, a collar, an item of clothing, a belt, a support, bedding, a blanket, a pillow, a cushion, a support surface of a seat, and a head-rest.

In certain embodiments, the enclosure has a configuration suitable for non-contact uses with the user, such as a structural building component, a post, a frame, etc.

In certain embodiments, the first sensor comprises one or more of a vibroacoustic sensor, a PPG/SpO2 sensor, and an electric potential sensor. The vibroacoustic sensor may comprise a voice coil type sensor which can detect frequencies in a range of about 0.1 Hz to about 160 kHz. The vibroacoustic sensor may have a diameter less than about 25 mm.

In certain embodiments, the second sensor comprises one or more of a pressure sensor, a temperature sensor, a humidity sensor, a light sensor, and an IMU.

In certain embodiments, one or both of the first substrate and the second substrate further comprises one or more of a communications component, a power source, a storage device, a processor, a USB port, a shield, a ground, and an amplifier. The amplifier may comprise a filter.

In certain embodiments, the foldable sensor device further comprises a battery sandwiched between the first substrate and the second substrate for providing power to one or both of the first sensor and the second sensor.

In certain embodiments, one or both of the first substrate and the second substrate includes a printed circuit board with one or both of the first sensor and/or the second sensor connected thereto.

In certain embodiments, the foldable sensor device further comprises a storage unit communicatively connected to one or both of the first sensor and the second sensor for storing data collected by the one or both of the first sensor and the second sensor.

In certain embodiments, one or both of the first sensor and the second sensor are configured to be communicatively connected to a processor of a computer system.

In certain embodiments, the processor is configured to receive signal data from the first sensor and/or the second sensor, and to determine a condition of a body in contact with or in proximity to the foldable sensor device based on the signal data.

In certain embodiments, on detection of a given condition, the processor is configured to execute one or more of the following steps: (i) cause a generation of an alarm; (ii) communicate with another device.

In certain embodiments, the processor is configured to receive signal data from the first sensor and/or the second sensor, and to determine whether there is an activity of a body in contact with or in proximity to the foldable sensor device based on the received signal data.

In certain embodiments, the processor is configured to receive signal data from the first sensor and/or the second sensor, and to identify an activity of a body in contact with or in proximity to the foldable sensor device based on the received signal.

In certain embodiments, the processor is configured to trigger, based on a data collection protocol, one or both of the first sensor and the second sensor to one or more of: start collecting data, stop collecting data, start storing the collected data and stop storing the collected data.

In certain embodiments, the first sensor and the second sensor are connected to a power source and wherein the data collection protocol is based on a consideration of balancing battery life with collection of pertinent data or storage of pertinent data.

In certain embodiments, the data collection protocol is based on a predetermined time interval. In certain embodiments, the data collection protocol is based on a predetermined time interval and a trigger event.

In certain embodiments, the trigger event comprises one or more of an intensity of a detected activity, an intensity of a detected signal compared to a threshold intensity, and a frequency of a detected signal compared to a threshold frequency.

In certain embodiments, one or both of the first substrate and the second substrate comprises at least one connector for connecting at least one additional substrate thereto.

In certain embodiments, the foldable sensor device further comprises a third substrate connected to the first substrate or to the second substrate by a second join member such that the second join member permits stacking of the first substrate, the second substrate and the third substrate. In other embodiments, there may be provided a fourth substrate and a third join member.

In certain embodiments, the first substrate, the second substrate and the third substrate are foldable so that the third substrate is sandwiched between the first substrate and the second substrate.

In certain embodiments, the first substrate and the second substrate are faces of a three-dimensional polyhedron which has been flattened to a two-dimensional form and re-folded.

In certain embodiments, a height of the foldable sensor device is less than about 15 mm high.

From another aspect, there is provided a wearable device comprising: an enclosure which can be placed against a body part of a user; at least one foldable sensor device, the foldable sensor device comprising: a first substrate having a first sensor; a second substrate having a second sensor; and a first join member connecting the first substrate and the second substrate such that the first substrate and the second substrate are folded relative to each other to form a folded configuration having multiple substrate layers stacked relative to one another.

In certain embodiments, the enclosure comprises one or more of: a strap, a band aid, a patch, a watch, a bandage, an item of jewelry, a head piece, an eye piece, an ear piece, a mouth piece, a collar, an item of clothing, a belt, a support, bedding, a blanket, a pillow, a cushion, a support surface of a seat, and a head-rest

From a yet further aspect, there is provided a sensing system comprising: a plurality of the foldable sensor device and/or the wearable devices, as described herein, and in which each foldable sensor device is in the folded configuration; and a processor of a computing system, the processor being communicatively connectable to each of the foldable sensor devices. In certain embodiments, a first foldable sensor device of the plurality of foldable sensor devices comprises a first enclosure which is a wearable enclosure, and a second foldable sensor device of the plurality of foldable sensor devices comprises a second enclosure which is a wearable enclosure and is different than the first enclosure. For example, the first enclosure may be a watch and the second enclosure may be a patch, both the first and second foldable sensor devices configured to be worn by the same user on the user's wrist and over the carotid artery, respectively.

In certain embodiments, the sensing system further comprises at least one additional sensor device for detecting signals associated to an environment around the additional sensor device, the processor communicatively connectable to the additional sensor device. The one additional sensor may comprise an environmental sensor, such as mounted outdoors for example to a building, post or gate.

In certain embodiments, the processor is configured to: receive signal data from each of the foldable sensor devices; determine from the received signal data whether an anomalous event has occurred in an environment in which any of the foldable sensor devices are positioned or to a user associated with any of the foldable sensor devices; cause an alert to be sent to a given user of a given foldable device for which the anomalous event has been determined, or cause an alert to be sent to an intelligence coordinator or microcontroller.

In certain embodiments, the processor is configured to determine the anomalous event by comparing the received signal data to baseline data relating to the environment and/or to the user.

From a further aspect, there is provided a method for manufacturing a foldable sensor device comprising: providing a first substrate and a second substrate connected to one another by a first join member, the first substrate and the second substrate forming an unfolded configuration, at least one of the first substrate and the second substrate having a at least one sensor associated therewith; and folding the first substrate relative to the second substrate to form a folded configuration in which the first substrate is stacked relative to the second substrate.

In certain embodiments, there is further provided a third substrate connected to one of the first substrate and the second substrate; wherein the unfolded configuration comprises one of the first substrate, the second substrate and the third substrate forming a central substrate, and wherein the folding comprises folding the other of the first substrate, the second substrate and the third substrate relative to the central substrate.

In certain embodiments, the unfolded configuration comprises the first substrate, the second substrate and the third substrate aligned linearly, and wherein the folding comprises folding at least one of the first substrate, the second substrate and the third substrate relative to an adjacent substrate.

In certain embodiments, providing the first substrate and the second substrate connected to one another by the first join member comprises selecting the first substrate and the second substrate from a plurality of different substrates based on an intended use, and joining the selected first substrate and the second substrate.

From a yet further aspect, there is provided a method of determining whether a user of a foldable sensor device has a condition, the method executed by a processor of a computer system communicatively coupled to at least one sensor of a foldable sensor device according to claim 1, the method comprising: receiving data related to the user from at least one sensor of the foldable sensor device; receiving data related to an environment of the user from at least one another sensor of the foldable sensor device; inputting the data to a machine learning algorithm; and obtaining an output of whether the user has the condition.

### Foldable Nature

Embodiments of the foldable sensor device for wearable uses are sized and shaped so that when it is in the folded configuration it can address epidermal surface and scale issues. Human skin has natural wrinkles, creases, and pits that are in the order of 15 μm to 100 μm. The foldable sensor device in its folded configuration may include sensors with a thickness in the order of natural skin creases. The enclosure of such a foldable sensor device may be sized to conform to small skin movements such as wrinkling, and present minimal obstructions to the user's skin sensations. The wearable device, in certain embodiments, may have a thickness smaller than or comparable to natural skin feature sizes. In this way the wearer will not feel his/her skin unnaturally restrained. Multifunction electronics, such as electric potential capacitive sensors that accurately detect noisy physiological signals, multilayer coils that enable on-skin RF communications, microfluidic volume and pressure sensors, and strain and hydration sensors that aid in post-operation recovery are included in these ultra-thin, foldable sensor solutions.

Certain embodiments of the folded wearable device comprise:
Invisible Design-Functional-folding to provide electronics greater skin contact without changing the user's behavior and/or sensory attunement to their own skin.
Optimal comfortable fabrication with materials that are thin and can ideally be folded to less than 100 μm to fit within natural wrinkles.
Reusable, recyclable and upcyclable, body-conforming, skin-conforming, unobtrusive, and can be comfortably worn for hours or days and then easily removed.
Durable-remain functional over typical usage of hours or days.
Offer discreet placement while also offering the possibility of bold, public aesthetically expressive placement.
Customizable to enable a broad adoption.
A hub to receive data from one or more devices and/or otherwise communicate with the one or more devices.

In certain embodiments, the present technology is integrated in an enclosure of a device. The device may be a wearable device, which may be a flexible wearable device. The device may comprise point-of-care and ambulatory patch solutions.

Embodiments of the present technology may harvest, relay and playback low frequency and low amplitude data generated by living and non-living subjects. In some aspects, the low frequency signals may comprise a bandwidth of between about 160,000 Hz and less than about 0.001 Hz, or about 24,000 Hz to about 0.01 Hz, or about 1000 Hz to about 1 Hz. In some other aspects, the low frequency signals may comprise a bandwidth of between about 20 Hz and less than about 1 Hz. In some other aspects, the low frequency signals may comprise a bandwidth of between about 15 Hz and less than about 0.1 Hz.

In certain embodiments, the device may be operated (assuming proper placement) on the skin, through clothes, over clothing and around obstacles.

In certain embodiments, the device may provide the operator with an indication (e.g. visual) of proper placement. In this respect, a positioning sensor may be provided to indicate adequate adherence of the patch to the subject.

In certain embodiments, the device may be able to transmit data through a traditional-looking oscilloscope or data reader that can be connected to the sensor array.

In certain embodiments, the system user, such as a clinician or a lay person, can use the system to record and store digital sound files.

The system may be configured to transmit the output with minimum delay (such as in real-time). In some aspects, the delay time will be less than about 10 seconds. In some aspects, the delay time will be less than about 100 milliseconds. In other aspects, the delay time will be less than about 10 milliseconds. In still other aspects, the delay time will be less than about 1 millisecond. In some aspects, the delay time will be less than about 100 microseconds. In other aspects, the delay time will be less than about 10 microseconds. In still other aspects, the delay time will be less than about 1 microsecond.

The device may comprise a manual and/or automatic on and off (button or switch).

The device may comprise an on/off indicator to signal the user as to whether the device is on or off.

The device may produce a distinct colored, steady light, when it is positioned properly on the body. Otherwise blinking (e.g. 1s) when on, and slower blinking (e.g. 5s) blinking when in stand-by mode. The “on” and “standby” modes may also be indicated by distinct colored lights.

The device may be usable world-wide, outdoors as well as indoors, under cover from rain.

In certain embodiments, the device comprises a rechargeable power source.

In certain embodiments, the device has a power source which allows for uninterrupted operation throughout an 8-hour shift without having to remember to power off the unit manually. In certain embodiments, the power source of the device can power the device for about 7-28 days, about, 21 days, about 18 days, about 14 days, about 13 days, about 12 days, about 11 days, about 10 days, about 9 days, about 8 days, about 7 days, about 6 days, about 5 days, about 4 days, about 3 days, about 2 days, about 24 hours, about 18 hours, about 12 hours or about 6 hours without recharging. A power indicator may be provided to indicate the need for recharging.

In certain embodiments, the device or the system is configured to save a volume and/or frequency mode when the device is turned off or powers down automatically.

In certain aspects, the technology may comprise a kit that includes a manual with instructions for setup, use, cleaning and maintenance, and troubleshooting.

According to certain embodiments, in the present technology, the devices, methods and systems may capture and process data relating to the subject such as one or more of:
Core body temperature of the subject
Heart audible/inaudible sounds of the subject
Lung audible/inaudible sounds of the subject
Gut audible/inaudible sounds of the subject
Central and peripheral vascular audible/inaudible sounds of the subject
Heart rate (plus variability) of the subject
Breath rate (plus variability) of the subject
Pulse and pulse delay & variability of the subject
Cough detection of the subject
Vocal changes of the subject
Stress and intensity of emotion state/emotional arousal changes of the subject via electrodermal activity (EDA)/galvanic skin response (GSR)
Continuous or intermittent blood pressure monitoring of the subject
Continuous or intermittent ballistocardiography
Continuous or intermittent seismocardiography
Blood oxygen content of the subject.

According to certain embodiments, in the present technology, the devices, methods and systems may capture and process data relating to the environment of the subject such as one or more of: Air temperature; Air humidity; Atmospheric pressure; Chemical composition; pollution; oxygen levels; carbon dioxide levels, carbon monoxide levels; Light conditions; Ecological disturbances and/or atmospheric disturbances such as those caused by directed energy attacks; and Noise conditions. Additionally, or alternatively, the devices and systems of the present technology may include an inertial measurement unit (IMU), a gyroscope, an accelerometer, and/or other suitable sensor(s) to characterize parameters associated with the environment.

In the context of the present specification, unless expressly provided otherwise, by animal is meant an individual animal that is a mammal, bird, or fish. Specifically, mammal refers to a vertebrate animal that is human and non-human, which are members of the taxonomic class Mammalia. Non-exclusive examples of non-human mammals include companion animals and livestock. Animals in the context of the present disclosure are understood to include vertebrates. The term vertebrate in this context is understood to comprise, for example fishes, amphibians, reptiles, birds, and mammals including humans. As used herein, the term “animal” may refer to a mammal and a non-mammal, such as a bird or fish. In the case of a mammal, it may be a human or non-human mammal. Non-human mammals include, but are not limited to, livestock animals and companion animals. As used herein, the term “plant” may refer to woody plants, such as trees, shrubs and other plants that produce wood as its structural tissue and thus has a hard stem. Other plants may include, but are not limited to, food crops such as grasses, legumes, tubers, leafy vegetables, brassica, root vegetables, gourd, fungi, pods and other seed, fruit, flower, bulb, stem, leaf and nut bearing crops.

In the context of the present specification, unless expressly provided otherwise, by non-living structures is meant buildings, machines, bridges, dams, geological formations, bodies of water, the atmosphere, etc.

In the context of the present specification, unless expressly provided otherwise, the terms “audible” and “inaudible” relate to sounds within the audible and inaudible range, respectively, of the average human ear. For example, inaudible sounds may have frequencies and/or amplitudes that cannot be heard by the average human ear and audible sounds may have frequencies and/or amplitudes that can be heard by the average human ear.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1A is a block diagram of an example system, including a multi-layer sensor device and a computing environment in accordance with various embodiments of the present technology;

FIG. 1B is a block diagram of an example computing environment in accordance with various embodiments of the present technology;

FIG. 2A is a first view of a foldable sensor device in accordance with various embodiments of the present technology;

FIG. 2B is a second view of the foldable sensor device in accordance with various embodiments of the present technology;

FIG. 3 is a folded configuration of the multi-layer sensor device in accordance with various embodiments of the present technology;

FIG. 4 illustrates assembly of a foldable sensor device in accordance with various embodiments of the present technology;

FIG. 5 illustrates the assembled foldable sensor device in accordance with various embodiments of the present technology;

FIG. 6 illustrates sensors of a foldable sensor device in accordance with various embodiments of the present technology;

FIG. 7 illustrates a first configuration of a foldable sensor device and an enclosure in accordance with various embodiments of the present technology;

FIG. 8 illustrates a second configuration of a foldable sensor device and an enclosure in accordance with various embodiments of the present technology;

FIG. 9 illustrates a third configuration of a foldable sensor device and an enclosure in accordance with various embodiments of the present technology;

FIG. 10 illustrates a fourth configuration of a foldable sensor device and an enclosure in accordance with various embodiments of the present technology;

FIG. 11 illustrates a watch with a foldable sensor device in accordance with various embodiments of the present technology;

FIG. 12 illustrates an unfolded configuration of a foldable sensor device with a central substrate;

FIG. 13 illustrates a folded configuration of the foldable sensor device with the central substrate;

FIG. 14 illustrates two views of example polyhedral shapes that may be used to form the substrates of the foldable sensor devices in accordance with various embodiments of the present technology;

FIG. 15 is a flow diagram of a method for collecting data in accordance with various embodiments of the present technology;

FIG. 16 is a flow diagram of a method for manufacturing a foldable sensor device in accordance with various embodiments of the present technology; and

FIG. 17 is a flow diagram of a method for predicting a medical condition of a user in accordance with various embodiments of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified embodiments of the present technology. As persons skilled in the art would understand, various embodiments of the present technology may be of greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and embodiments of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry and/or illustrative systems embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general purpose processor, such as a central processing unit (CPU) or a processor dedicated to a specific purpose, such as a digital signal processor (DSP). Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Some or all of the functions described herein may be performed by a cloud-based system. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown. Moreover, it should be understood that one or more modules may include for example, but without being limitative, computer program logic, computer program instructions, software, stack, firmware, hardware circuitry, or a combination thereof.

### Systems

### Computing Environment

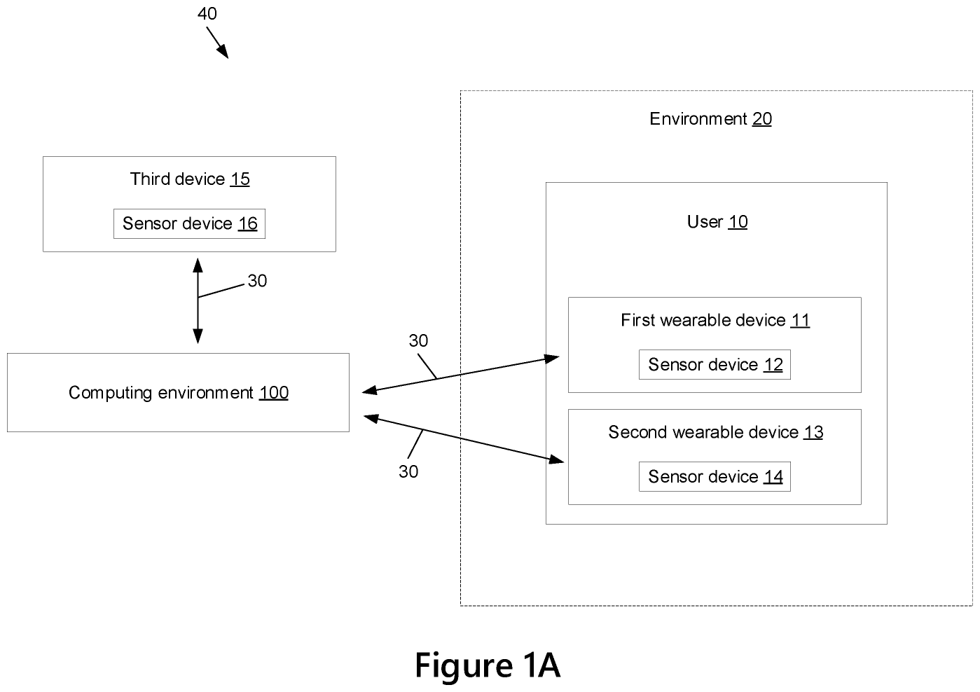

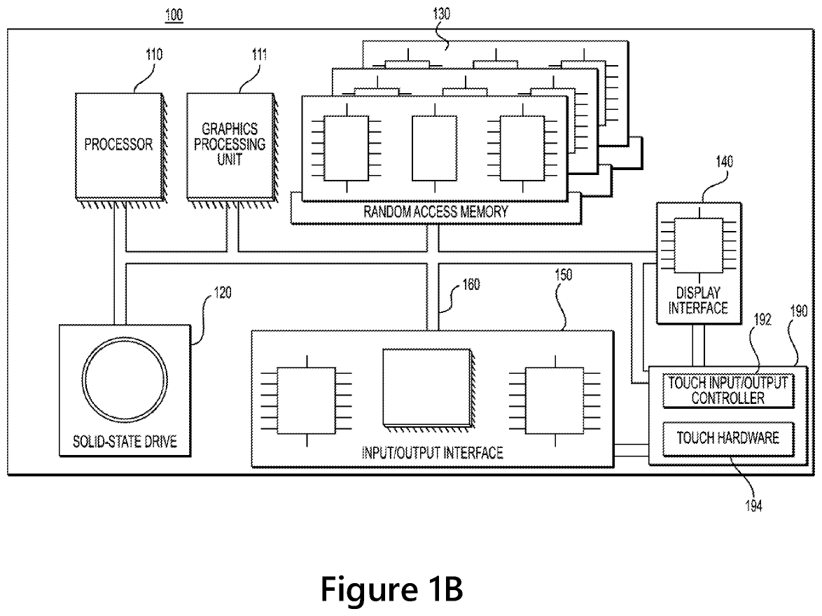

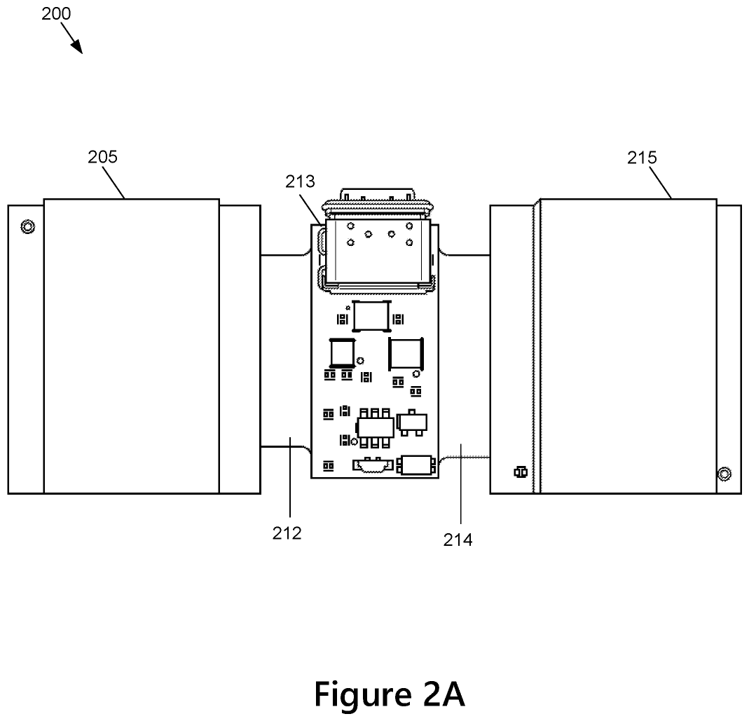

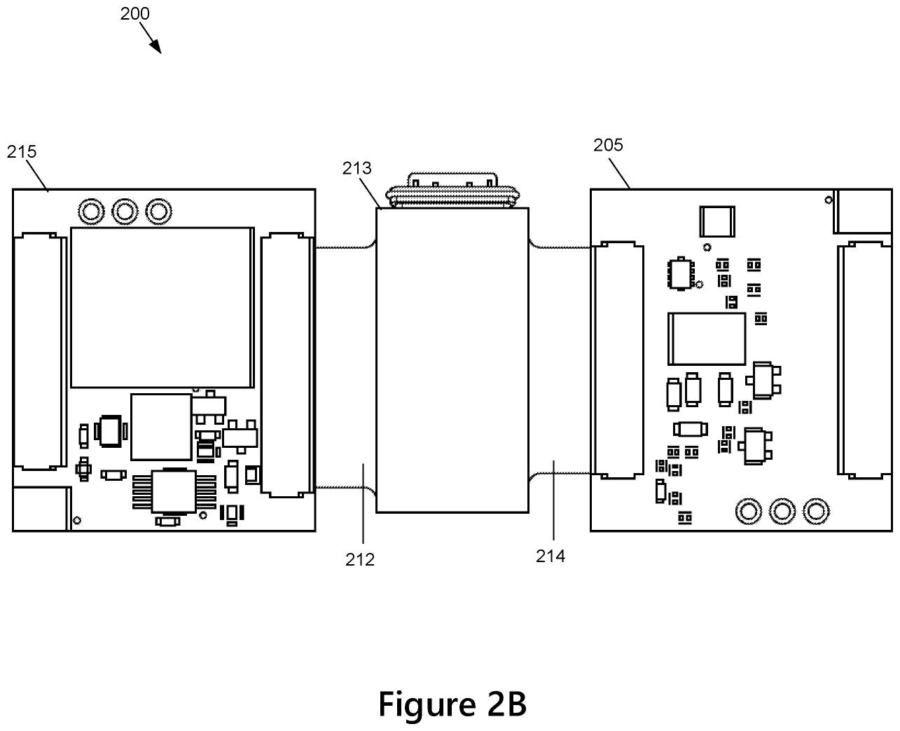

FIG. 1A illustrates a system 40 for implementing and/or executing any of the devices and/or methods described herein such as for example monitoring, enhancing, preventing or predicting a condition of a user 10 in an environment 20. In certain embodiments, the system comprises a first wearable device 11, including one or more sensors in a sensor device 12, a second wearable device 13, including one or more sensors in a sensor device 14, and/or a third device 15 including one or more sensors in a sensor device 16. The first wearable 11, second wearable device 13, and/or third device 15 are communicatively coupled to a processor 110 of a computing environment 100 via a network 30. The sensor devices 12, 14, and/or 16 may comprise a multi-layer sensor device 12 which will be described later with reference to FIGS. 2-11.

The first wearable device 11 and/or second wearable device 13 may be worn by the user 10. For example the first wearable device 11 may be a watch and the second wearable device 13 may be a flexible patch. The sensor devices 12 and 14 may record data about the user 10 and/or the environment 20 surrounding the user 10. The third device 15 may be positioned within the environment 20. For example the third device 15 may be attached to a building, tower, and/or other structure. The sensor device 16 may contain sensors that measure the environment 20 surrounding the user 10. The first wearable device 11, second wearable device 13, and/or third device 15 may simultaneously record data about the user 10 and/or environment 20. Timestamped data may be collected from each of the first wearable device 11, second wearable device 13, and/or third device 15. A location of each of the first wearable device 11, second wearable device 13, and/or third device 15 may be determined. A distance between each of the first wearable device 11, second wearable device 13, and/or third device 15 may be determined, such as by measuring the time-of-flight of communications transmitted between the devices.

In other embodiments (not shown), there may be provided the first wearable device 11 including the one or more sensors in the sensor device 12, without provision of the second wearable device 13 and the third device 15. The computing environment 100 may be a standalone device (as illustrated) and/or integrated within the first wearable device 11, second wearable device 13, and/or third device 15. The computing environment 100 may be integrated in an intelligence coordinator device (e.g. a microcontroller). The intelligence coordinator device may gather data from multiple sensor devices and/or other devices. Individual devices may send alerts to the intelligence coordinator device, such as after detecting an anomalous event.

In yet other embodiments (not shown), there may be provided additional wearable devices or other devices with sensors communicatively coupled to the processor 110 of the computing environment 100 via the network 30.

The environment 20 may include the user 10 and/or other users (not illustrated). Data about the other users and/or about the environment may be collected, such as by wearable devices being worn by the other users. Data collected about the other users in the environment 20 may be collected by the computing environment 100 and processed as environmental data corresponding to the user 10. In other words, the data collected from the other users in the environment 20 may be used as data describing the environment 20.

Data collected from the sensors, such as the third device 15, may identify multiple individuals in the environment 20, such as all individuals present in the environment 20. The computing environment 100 may determine, based on collected sensor data, which of the individuals in the environment 20 are wearing a wearable device. All individuals associated with an organization, such as a military, may be wearing wearable devices. The computing environment 100 may identify which individuals in the environment 20 are not wearing wearable devices and/or associated with the organization. The computing environment 100 may display an interface, such as a map, showing locations of all individuals in the environment and/or an indication for each individual showing whether the individual is associated with the organization or not.

FIG. 1B illustrates an embodiment of the computing environment 100. In some embodiments, the computing environment 100 may be implemented by any of a conventional personal computer, a network device and/or an electronic device (such as, but not limited to, a mobile device, a tablet device, a server, a controller unit, a control device, etc.), and/or any combination thereof appropriate to the relevant task at hand. In some embodiments, the computing environment 100 comprises various hardware components including one or more single or multi-core processors collectively represented by processor 110, a solid-state drive 120, a random access memory 130, and an input/output interface 150. The computing environment 100 may be a computer specifically designed to operate a machine learning algorithm (MLA). The computing environment 100 may be a generic computer system.

In some embodiments, the computing environment 100 may also be a subsystem of one of the above-listed systems. In some other embodiments, the computing environment 100 may be an “off-the-shelf” generic computer system. In some embodiments, the computing environment 100 may also be distributed amongst multiple systems. The computing environment 100 may also be specifically dedicated to the implementation of the present technology. As a person in the art of the present technology may appreciate, multiple variations as to how the computing environment 100 is implemented may be envisioned without departing from the scope of the present technology.

Those skilled in the art will appreciate that processor 110 is generally representative of a processing capability. In some embodiments, in place of or in addition to one or more conventional Central Processing Units (CPUs), one or more specialized processing cores may be provided. For example, one or more Graphic Processing Units 111 (GPUs), Tensor Processing Units (TPUs), and/or other so-called accelerated processors (or processing accelerators) may be provided in addition to or in place of one or more CPUs.

System memory will typically include random access memory 130, but is more generally intended to encompass any type of non-transitory system memory such as static random access memory (SRAM), dynamic random access memory (DRAM), synchronous DRAM (SDRAM), read-only memory (ROM), or a combination thereof. Solid-state drive 120 is shown as an example of a mass storage device, but more generally such mass storage may comprise any type of non-transitory storage device configured to store data, programs, and other information, and to make the data, programs, and other information accessible via a system bus 160. For example, mass storage may comprise one or more of a solid state drive, hard disk drive, a magnetic disk drive, and/or an optical disk drive.

Communication between the various components of the computing environment 100 may be enabled by a system bus 160 comprising one or more internal and/or external buses (e.g., a PCI bus, universal serial bus, IEEE 1394 “Firewire” bus, SCSI bus, Serial-ATA bus, ARINC bus, etc.), to which the various hardware components are electronically coupled.

The input/output interface 150 may allow enabling networking capabilities such as wired or wireless access. As an example, the input/output interface 150 may comprise a networking interface such as, but not limited to, a network port, a network socket, a network interface controller and the like. Multiple examples of how the networking interface may be implemented will become apparent to the person skilled in the art of the present technology. For example the networking interface may implement specific physical layer and data link layer standards such as Ethernet, Fibre Channel, Wi-Fi, Token Ring or Serial communication protocols. The specific physical layer and the data link layer may provide a base for a full network protocol stack, allowing communication among small groups of computers on the same local area network (LAN) and large-scale network communications through routable protocols, such as Internet Protocol (IP).

The input/output interface 150 may be coupled to a touchscreen 190 and/or to the system bus 160. The touchscreen 190 may be part of the display. In some embodiments, the touchscreen 190 is the display. The touchscreen 190 may equally be referred to as a screen 190. In the embodiments illustrated in FIG. 1B, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the system bus 160. The display interface 140 may include and/or be in communication with any type and/or number of displays. In some embodiments, the input/output interface 150 may be connected to a keyboard (not shown), a mouse (not shown) or a trackpad (not shown) allowing the user to interact with the computing device 100 in addition to or instead of the touchscreen 190.

According to some embodiments of the present technology, the solid-state drive 120 stores program instructions suitable for being loaded into the random access memory 130 and executed by the processor 110 for executing acts of one or more methods described herein. For example, at least some of the program instructions may be part of a library or an application. Some or all of the components of the computing environment 100 may be integrated in a multi-layer sensor device and/or in communication with the multi-layer sensor device. The processor may be configured to process the data obtained by the multi-layer sensor device, and provide an output, such as to a smartphone of an operator of the system.

### Sensors

Present systems and devices, in certain embodiments, include sensors for capturing, and optionally processing, data associated with a user or of an environment of the user.

The one or more sensors used in the present technology is not particularly limited. The one or more sensors used in the present technology may include sensor stacks and/or other plug-and-play devices and/or systems.

In certain embodiments, the one or more sensors comprise a sensor array. The sensors may be selected from:
Passive and/or active vibrometry and vibroacoustic sensors. In certain embodiments, the sensor is a vibroacoustic sensor for detecting vibroacoustic signals. In some embodiments, transmission of vibroacoustic waves may occur through an intermediate medium such as air.

In some embodiments, the vibroacoustic sensor may have a bandwidth suitable for detecting vibroacoustic signals in the infrasound range, such as a bandwidth ranging from about 0.01 Hz to at least about 20 Hz. Furthermore, in some embodiments, the vibroacoustic sensor may have wider bandwidths covering a wider spectrum of infrasound-to-ultrasound, such as a bandwidth ranging from about 0.01 Hz to at least 160 kHz. In some embodiments, the biological vibroacoustic signal component extracted from the detected vibroacoustic signal may have a bandwidth ranging from about 0.01 Hz to 0.1 Hz.

For example, in some embodiments the vibroacoustic sensor may have an overall bandwidth ranging from about 0.01 Hz to at least about 50 kHz, from about 0.01 Hz to at least about 60 kHz, from about 0.01 Hz to at least about 70 kHz, from about 0.01 Hz to at least about 80 kHz, from about 0.01 Hz to at least about 90 kHz, from about 0.01 Hz to at least about 100 kHz, from about 0.01 Hz to at least about 110 kHz, from about 0.01 Hz to at least about 120 kHz, from about 0.01 Hz to at least about 130 kHz, from about 0.01 Hz to at least about 140 kHz, from about 0.01 Hz to at least about 150 kHz, from about 0.01 Hz to at least about 160 kHz, from about 0.01 Hz to more than about 150 kHz.

The sensor may, in some embodiments, comprise a single sensor that provides one or more of the abovementioned bandwidths of detected vibroacoustic signals.

In some other embodiments, the sensor may include a suite or array of multiple sensors, each having a respective bandwidth range forming a segment of the overall vibroacoustic sensor bandwidth. At least some of these multiple sensors may have respective bandwidths that at least partially overlap in certain embodiments. In other embodiments, the multiple sensors do not have overlapping bandwidth ranges. Accordingly, various sensor bandwidths may be achieved based on a selection of particular sensors that collectively contribute to a particular vibroacoustic sensor bandwidth. In other words, bandwidth extension and linearization approach (bandwidth predistortion) may utilize modular sensor fusion and response feedback information, such as to compensate for bandwidth limitations of any particular single sensor with overlapped combinations of sensors to cover a wider bandwidth with optimal performance.

Such sensors may be used for detecting chronic pulmonary disease, lung consolidation, diffuse alveolar damage, vascular injury, and/or fibrosis, pulmonary embolism, and disseminated intravascular coagulation (DIC) cardiac structural disorders such as left ventricular hypertrophy, carotid disease, coronary disease, to name a few.

Certain example sensors for passive and/or active vibroacoustic sensors are described in PCT/US2021/046566 filed Aug. 18, 2021, the contents of which are herein incorporated by reference. In certain embodiments, passive vibroacoustic sensors are used which can detect frequencies of about 0.1 Hz to about 160 kHz. In certain embodiments, active vibroacoustic sensors are used which can detect frequencies of about 10 Hz to about 10 GHz.

More specifically, in certain embodiments, one or more of the sensors comprises a vibroacoustic transducer of a voice coil type. The voice coil transducer comprises a magnet housing having a cylindrical body portion with a bore and a flange extending radially outwardly from the cylindrical body portion. An iron core such as soft iron or other magnetic material is attached to the cylindrical body portion and lines the bore of the cylindrical body portion. The iron core extends around the bore of the cylindrical body portion as well as across an end of the cylindrical body portion. The iron core has an open end. A magnet is positioned in the bore and is surrounded by, and spaced from, the iron core to define a magnet gap. A voice coil, comprising one or more layers of wire windings supported by a coil holder, is suspended and centered in relation to the magnet gap by one or more spiders. The wire windings may be made of a conductive material such as copper or aluminum. A periphery of the spider is attached to the frame, and a center portion is attached to the voice coil. The voice coil at least partially extends into the magnet gap through the open end of the iron core. The one or more spiders allow for relative movement between the voice coil and the magnet whilst minimizing or avoiding torsion and in-plane movements. The voice coil transducer is attached to a diaphragm.

Additionally, the voice coil transducer is attached to the frame by the support members. Rotational movement of the frame relative to the frame is limited. Movements induced in the acoustic waves will cause the diaphragm to move, in turn inducing movement of the voice coil within the magnet gap, resulting in an induced electrical signal.

In certain variations of the voice coil transducer, the configuration of the transducer is arranged to pick up more orthogonal signals than in-plane signals, thereby improving sensitivity. For example, the one or more spiders are designed to have out-of-plane compliance and be stiff in-plane. The same is true of the diaphragm whose material and stiffness properties can be selected to improve out-of-plane compliance. The diaphragm may have a convex configuration (e.g. dome shaped) to further help in rejecting non-orthogonal signals by deflecting them away. Furthermore, signal processing may further derive any non-orthogonal signals e.g. by using a 3 axis accelerometer. This either to further reject non-orthogonal signals or even to particularly allow non-orthogonal signals through the sensor to derive the angle of origin of the incoming acoustic wave.

It will be appreciated that different uses and different sizes of the sensing device may require different sensitivities and face different noise/signal ratios challenges. For example, higher sensitivity and increased signal/noise ratio will be required for clothing contact uses compared to direct skin contact uses. Similarly, higher sensitivity and increased signal/noise ratio will be required for non-contact uses compared to contact uses. Therefore, in order to provide sensing devices having sensitivities and signal/noise ratios suitable for different form factors (e.g. contact or non-contact uses), developers have discovered that modulation of certain variables can optimize the voice coil transducer for the specific intended use: magnet strength, magnet volume, voice coil height, wire thickness, number of windings, number of winding layers, winding material (e.g. copper vs aluminum), and spider configuration. Developers also discovered that adaptation of the configuration of the spider contributed to increasing sensitivity and signal/noise ratio increases. More specifically, it was determined via experiment and simulation that making the spider more compliant such as by incorporating apertures in the spider, increased sensitivity. Apertures also allow for free air flow. In certain embodiment, the sensor comprises a voice coil transducer with the properties listed below.

| | Present | | Present | |
|---|---|---|---|---|
| Parameter | technology 1 | | technology 2 | |
| Impedance | 150 ohms ± 2% | | 150 ohms ± 2% | |
| DC Resistance (Re) | 150 ohms ± 2% | | 150 ohms ± 2% | |
| Voice Coil | 7.5 mH at 1 kHz/2.5 | | 8.46 mH at 1 kHz/2.7 | |
| Inductance (Le) | mH at 10 kHz | | mH at 10 kHz | |
| Coil Resonant | 80-170 | Hz | 90 Hz ± 2% | |
| Frequency (Fs) | | | | |
| Total Q (Qts) | 0.25 to 0.65 | | 0.85-0.90 | |
| Inverse of | (depending on | | | |
| damping | exciter)/100 | | | |
| | mg to 100 g | | | |
| | depending on | | | |
| | no. windings) | | | |
| Moving Mass | 100 mg to 100 g | | 1.15 | g |
| (Mms) | (depends on number | | | |
| | of windings) | | | |
| | For test exciters | | | |
| | specifically 1.15 g | | | |
| Mechanical Compliance | 0.4 to 3.2 | mm/N | 3.2 | mm/N |
| of Suspension (Cms) | | | | |
| (inverse of suspension) | | | | |
| BL Product (BL) | 18.5 N/Amp | | 18.5 | Tm |
| | (same as Tm) | | | |
| Voice Coil Diameter | 25 | mm | 25 | mm |
| RMS Power Handling | 2 | W | 2 | W |
| Wire Diameter | 0.05 | mm | 0.05 | mm |
| Number of windings | 208 | | 208 | |
| Number of Layers | 4 | | 4 | |
| Magnet Size | 24 mm × 3.5 mm | | 24 mm × 3.5 mm | |
| Overall Outside | 50.5 mm (5 × 5 × 0.1 | | 60 mm and 65 mm | |
| Diameter | to 50 × 50 × 10) | | (oval shaped) | |
| Overall Depth | 20.5 | mm | 27 | mm |
| Inductance/moving | at least 6.52 mH | | 7.36 mH per gram | |
| mass ratio | per gram at 1 kHz | | at 1 kHz | |
| Mechanical compliance/ | at least 0.348 | | 2.78 mm/N | |
| moving mass ratio | mm/N per gram | | per gram | |
| BL product/moving | at least 16 N/Amp | | 16.09 N/Amp | |
| mass ratio | per gram | | per gram | |
| (BL × mechanical | 51.48 | | 51.48 | |
| compliance)/ | [T*m\hat{ }2/(N * g)] | | [T*m\hat{ }2/(N * g)] | |
| moving mass | | | | |
| Wright Parameters | | | | |

| | | |
|---|---|---|
| K(r) | 26-27 | 23 |
| X(r) | 0.175-0.185 | 0.194 |
| K(i) | 0.00709-0.01118 | 0.032 |
| X(i) | 0.827-0.866 | 0.739 |

Bio-electric sensors, such as multi-lead ECG. Such sensors may be used for detecting conduction block, ventricular tachycardia, and/or ventricular fibrillation, cardiovascular disease, hypertension, diabetes. The most common complications include arrhythmia (atrial fibrillation, ventricular tachyarrhythmia, and ventricular fibrillation), cardiac injury, heart failure. QT prolongation, etc., for example, Changes in the QT interval in general-care patients who are undergoing treatment with drugs that can prolong QT intervals and may cause life threatening arrhythmias. On certain embodiments, widefield capacitive electric potential sensors are used. The bio-electric sensors may be able to record measurements with and/or without physical or resistive coupling. The bio-electric sensors may be configured to obtain Electrocardiogram (ECG), Electroencephalogram (EEG), Electrooculograph (EOG), and/or Electromyograph (EMG) readings. The bio-electric sensors may be used for imaging, security, and human-machine interfaces and devices that can “see” through walls.
Bio-electric sensors such as sensors for detecting Galvanic Skin Response (GSR), also referred to as Electrodermal Activity (EDA)/Electrodermal Response (EDR) or Skin Conductance (SC), and Psychogalvanic Reflex (PGR)-rapidly changing physiological characteristics of the largest organ of the human body—the skin. The skin functions as the principal interface between organism and environment. Together with other organs, it is responsible for bodily processes such as the immune system, thermo-regulation, and sensory-motor exploration.
Bio-electric sensors such as a non-contact electroencephalography (EEG) device, can detect a state of a person in proximity to the non-contacting sensor. The detected state may include an emotional state, a cognitive load state, and/or an alertness state of the person in proximity to the non-contacting sensor. When a processor in communication with and/or integrated within the non-contact device detects a pattern corresponding to a predetermined state of the person in proximity to the non-contacting sensor, the processor can transmit an action signal to another device to take a subsequent action, such as displaying an alert and/or or generating a “smart-alarm”.
Oxygen saturation sensors, such as Red+Green LED-O₂ saturation or Red LED plus IR LED-Normal oxygen levels are at least 95%. Some patients with chronic lung disease or sleep apnea can have normal levels around 90%. Oxygen saturation is a crucial measure of how well the lungs are working. Oxygen saturation measured by pulse oximetry is most frequently used to monitor patients with lung and heart disorders, who are at risk of low levels of blood oxygen. Oxygen saturation is also used to monitor patients before, during, and after surgery, including during anesthesia; to monitor patients on certain medications that may reduce respiration and lung function; to assess the lung function of people with conditions that can cause reduction of blood oxygen levels, including COPD, asthma, acute respiratory distress syndrome (ARDS), anemia, pneumonia, lung cancer, cardiac arrest, and heart failure, among others; to assess individuals with sleep disorders such as sleep apnea.
Aroma, gas and smell sensors. These e-nose solutions can include optical, surface acoustic wave (SAW), electrochemical, capacitive, catalytic, semiconductor gas sensors, electrical variation methods, imprinted polymer sensors, etc. rely on the following substances as a sensing.

Ultrasonic based acoustic sensors are principally classified as (1) ultrasonic, (2) attenuation, and (3) acoustic impedance. The major method for detection of sound velocity is to determine the time-of-flight that measures the travel time of ultrasonic waves at a known distance to calculate their speed of propagation. The measured gas speed is used for (1) identification of gases by determining gas properties such as gas concentration, which is related to the difference of sound propagation time, and for (2) determining the components or the molar weight of various gases in mixtures proceeding from thermodynamic considerations. Generally, ultrasonic sensors can overcome some shortcomings of gas sensors such as short lifetime.

Ecological, atmospheric, and environmental infrasound-to-terahertz sensors may be included in a broad-based, multi-focal, and/or adaptable technology solution. These sensors may be used to detect, identify, and/or defend against infrasound-to-ultrasound, electromagnetic frequency (EM), radio frequency (RF), laser, and/or magnetic resonance yoked instruments.

Attenuation is the energy loss due to thermal losses and scattering when an acoustic wave propagates through a medium. Each gas demonstrates particular attenuation, therefore providing means to determine target gases. Gas attenuation can be utilized together with sound velocity to determine gas properties. However, the attenuation method is not so reliable as the method of sound speed because it is prone to the presence of particles and droplets or the turbulence in the gas.

Acoustic impedance is typically employed for assessment of gas density. Therefore, by the quantified acoustic impedance and speed of sound, the density of a gas could be found out. In any case, the quantification of the acoustic impedance of gases is remarkably troublesome, particularly in a process environment and consequently it is rarely used in practice.

Semiconductor gas sensing depends on the variation of the oxide on the surface of a metal-oxide-semiconductor (MOS) stack which is transformed into a change of the sensor's electrical resistance as a means to detect gases via redox reactions between the target gas(es) and the oxide surface. To obviate the high operating temperature requirement, which can limit their application, microheaters produced by VLSI CMOS technology are used. Another issue, the relatively lengthy time needed for the gas sensor to recover after each gas exposure, which is impractical for applications where gas concentration changes quickly, is overcome by using nanodimension structures (e.g., nanowires and nanotubes).

Polymer-based sensors detect aromas and gases released by the body using a polymer layer that is changes its physical properties (mass, dielectric properties) upon gas absorption. Polymer sensors detect volatile organic compounds such as alcohols, formaldehyde, aromatic compounds or halogenated compounds released by skin and breath. Polymer gas sensors possess benefits such as high sensitivities and short response times. Their shortcomings include lack of long-term stability, reversibility and reduced selectivity.

Carbon nanotube sensors overcome the problem of insufficient sensitivity at room temperature observed by MOS sensors. The properties of carbon nanotubes (CNTs) allow the development of high-sensitive gas sensors. CNT sensors demonstrate ppm levels response for a range of gases at room temperature, which makes them perfect for low power applications. Their electrical properties carry high sensitivity to very small quantities of gases such as carbon dioxide, nitrogen, ammonia, oxide, and alcohol at room temperature (unlike MOS sensors, which should be heated by a supplementary heater in order to operate normally). Multiwall CNTs have been employed for remote sensing of carbon dioxide (CO2), ammonia (NH3), and oxygen (O2). To enhance selectivity and sensitivity of sensing, CNTs are often combined with other materials. Moisture absorbing materials detect moisture, because their dielectric constant can be tuned to be altered by the water content in the environment. They can be used also as a substrate of the devices of the present technology because the dielectric constant of moisture absorbing materials can be regulated by the moisture of the neighboring air.

Gas sensing can also be achieved based on variation of non-electrical properties like optical, calorimetric, gas chromatograph, and acoustic sensing. Optical sensors rely on spectroscopy, which uses emission spectrometry and absorption. The principle of absorption spectrometry is based on absorption of the photons at specific gas wavelengths; the absorption depends on the concentration of photons. Infrared gas sensors operate on the principle of molecular absorption spectrometry; each gas has its own particular absorption properties to infrared radiation with different wavelengths. In general, optical sensors could attain better selectivity, sensitivity, and stability in comparison to non-optical methods.

calorimetric sensors are solid-state devices. The sensitive elements consist of small ceramic “pellets” with varying resistance depending on the existence of target gases. They detect gases with a substantial variation of thermal conductivity with reference to the thermal conductivity of air (e.g., combustible gases). Gas chromatography is a classic analytical method with exceptional capabilities for separation as well as high selectivity and sensitivity. However, gas chromatograph miniaturization is challenging. Other sensors which can be incorporated in the present technology are illustrated below.

| | | Physiological/ |
|---|---|---|
| Sensor | Environmental | Biometric |
| GPS [lat/lon to supported precision (e.g. roughly 64 bits/sample)] | x | |
| 9-axis IMU [e.g. 16-bit samples × 9 (XYZ on three sensors). | x | X |
| 10-100 Hz sample rate when active.] | | |
| Humidity [e.g. 8-bit samples, 0.1 Hz sample rate.] | x | |
| Barometric Pressure [e.g. 24-bit samples, 50 Hz sample rate.] | x | X |
| Ambient Temperature [May be included in pressure sensor. E.g. 8-bit | x | |
| samples (0.5° F. resolution). 0.1 Hz sample rate.] | | |
| Core body Temperature (e.g. non-contact infra-red body temperature | | X |
| sensor, or a thermometer or a thermography sensor) | | |
| MEMS Microphone [e.g. Single microphone because no significant directionality | x | X |
| in this form factor with multiple mics because of limited spacing. | | |
| 16-bit sample rate, 48 kHz sample rate, inline compression.] | | |
| Ambient Light [e.g. Single band (eye spectral response) and 8-bit | x | |
| samples (log scale), 0.1 Hz sample rate.] | | |
| Ionization radiation detector [Csl: TI scintillator, personal dosimeter, | x | |
| 8-bit samples (log scale), e.g. 0.1 Hz sample rate.] | | |
| Radiofrequency and Terahertz submillimeter radiation sensor stack (e.g. field | | X |
| strength sensor in the range of about 1 kHz to about 50 GHz or Passive RF | | |
| energy burst (or modulated) in the 10 MHz-6 GHz range) | | |
| Pulse rate, respiratory rate, body temperature, blood pressure, and peripheral | | X |
| capillary oxygen saturation [4 × 4 red, green, and blue (RGB) LED array 12-bit | | |
| samples, 100 Hz sample rate.] | | |
| Electric potential sensor [ECG and AC bioimpedance, skin conductance, galvanic | | X |
| skin response (GSR), electrodermal response (EDR), electrodermal activity (EDA), | | |
| 12-bit samples, 16 kHz sample rate] | | |
| Ballistocardiography vibroacoustic and physiological and environmental infrasound | x | X |
| sensor [far infrasound-to-far ultrasound physiologic sensor, 16-bit samples, 48 kHz | | |
| sample rate, pre-compression . . .] | | |

Other sensors include vO2, vCO2, respiratory quotient (RQ), and energy expenditure (EE), B-2 & B-52 sorties: ketones and hydration, CO2 excretion, tidal capnography.
SpO2, SaO2, PaO2 sensor suite.
Integrated Biopotential and Bioimpedance AFE.
Low-Power, Integrated In-Ear Heart-Rate Monitor.
Finger Heart Rate and Pulse Oximeter Smart Sensor.
Thermistor on flex foil (temperature sensor).
Non-contact infrared temperature sensor.
Optical Pulse Oximeter and Heart-Rate Sensor.
Health Sensor (hSensor) platform supporting the measurement of motion, precision skin temperature, bio-potential measurements (including ECG, EMG, EEG, and DC-potential EEG) and reflective PPG (including pulse oximetry and heart rate).
GSR.
Ballistocardiography (BCG) and seismocardiography (SCG) sensors.
DC-potential electromagneticcardiogram (EMCG) sensor.
Circadian rhythm sensor.
Ultra-Wide-Band sensor breathing analyzer.
Electrochemical Sensor AFE.
Continuous and/or pulsed Infrasound-to-ultrasound.
Continuous and/or pulsed electromagnetic frequency (EM).
Continuous and/or pulsed radio frequency (RF).
Continuous and/or pulsed lo/hi energy laser.
Continuous and/or pulsed magnetic resonance.

The standard electrocardiogram (ECG), measures the electrical activity of heart and is widely used in clinical settings, is difficult to integrate into wrist worn devices. Instead, most current solutions focus on photoplethysmography (PPG), which operates by shining light onto the body and measuring the amount of reflection which is modulated by the blood flow. This means that flow, volume, pressure, etc., factors and morphological components of the waveform (such as P-waves and T-waves in the ECG and heart rate variability cannot be directly reported and are indirectly inferred using biophysiology-independent black-box AI algorithms. Moreover, as PPG sensing uses a light source (typically an LED), it inherently consumes a large amount of power.

Integration of ECG into a wearable device such as standard watch at the wrist is challenging due to the need for electrodes to be placed on either side of the heart. If electrodes are placed on just one side of the heart, the collected signals reduce in amplitude and become increasingly small the further away from the heart. Placing electrodes on just one arm, the time domain ECG signal reaches a 0 dB Signal-to-Noise Ratio at (approximately) the elbow. It is possible, however, to place one electrode on one wrist, and then touch a second electrode with the other hand as done with the Apple™ watch. As the two sensing connection points are on either side of the heart a high Signal-to-Noise Ratio (SNR) ECG can be collected.

Sensors to be included in the foldable sensor device may include any other sensor that can monitor vibroacoustic and electrical activity generated by the body and/or brain of a user without or without making contact with the body.

### Devices

### Foldable Sensor Device in an Unfolded Configuration

From a broad aspect, the sensor device 12 of FIG. 1 comprises a plurality of sensors supported by two or more substrates and which can have a folded configuration or an unfolded configuration. In the folded configuration, the sensor device 12 has multiple substrate layers supporting the plurality of sensors. The multiple layers of the sensor device 12 may be formed through a folding mechanism.

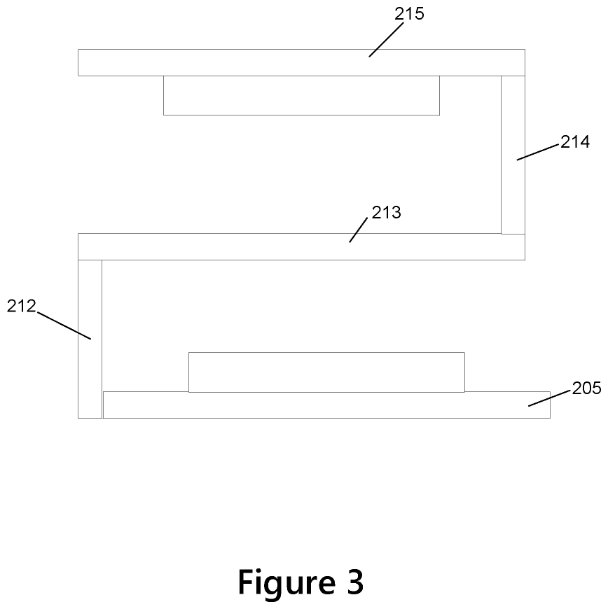

Certain embodiments of the sensor device 12 are illustrated in FIGS. 2A and 2B as a foldable sensor device 200, when in the unfolded configuration. In the folded configuration (not shown), the foldable sensor device 200 will have three layers stacked one above another. The foldable sensor device 200 includes substrate 205, a join member 212, a substrate 213, a join member 214, and a substrate 215. The join members 212 and 214 permit the substrate 205 and the substrate 215 to be folded relative to each other into the folded configuration. Sensors and/or other electronic components may be positioned on one or more of the substrates 205, 213, and 215. The substrates 205, 213, and/or 215 may include printed circuit boards (PCBs) and the sensors may be attached to the PCBs. It will be appreciated that for a given substrate, the sensors may be positioned on one or both sides of the substrates. FIGS. 2 and 3 show opposing sides of the foldable sensor device 200 and in the embodiment illustrated the substrate 213 has sensors/electronic components on a side opposite to that of the substrates 205, 215.

In use, the foldable sensor device 200 is placed against, or close to, a body part of a user, such as the user's skin, when in the folded configuration with one side of the substrates 205, 215 facing the body part.

All or a portion of the join members 212 and 214 may be flexible and/or formed of a flexible material. Alternatively, or additionally, the join members 212 and 214 may form a hinge, accordion-like structure, and/or any other suitable structure for allowing the substrate 205 and substrate 215 to be positioned in a multi-layer configuration. The join members 212 and/or 214 may be thinned or otherwise dimensioned to enable the foldable sensor device 200 to be folded.

One or more of the substrates 205, 213, 215 may be made of the same material as the join members 212, 214 and include a stiffener material for strengthening and rendering less flexible. For example, as illustrated in FIG. 2, the substrate 213 includes the stiffener (rectangular portion seen in FIG. 2). In other embodiments, one or more of the substrates 205, 213, 215 may be made of a different material than the join members 212, 214. For example, the one or more of the substrates 205, 213, 215 may be made of a stiffer material than the join members 212, 214.

Advantageously, in certain embodiments, the substrate 205, join member 212, substrate 213, join member 214, and/or substrate 215 may be manufactured as a single piece. In other embodiments, the substrates and the join members may comprise different components which are attached to each other.

FIGS. 2A and 2B illustrate an unfolded configuration of the foldable sensor device 200. In this unfolded configuration, the substrate 205, join member 212, substrate 213, join member 214, and/or substrate 215 are co-planar. In the folded configuration (see FIG. 3), the substrate 205 and substrate 215 may be stacked one above each other.

The arrangement of the sensors on the substrates 205, 213, and 215 may be such that in use, when the foldable sensor device 200 is positioned on or near the body of the subject, some of the sensors of the foldable sensor device 200 may face the user's body and/or some of the sensors of the foldable sensor device 200 may face outwardly towards the environment, away from the user's body. The sensors facing towards the user's body may capture physiological data of the user. The sensors facing away from the user's body may capture environmental data describing the environment surrounding the user. Both types of data, physiological and environmental, may be captured simultaneously by the foldable sensor device 200. The data capture may be continuous or intermittent.

In other embodiments (not shown), at least a portion of at least one sensor may be formed within the body of the substrate 205, substrate 213, and/or substrate 215. Such sensor or sensor portion may include filtering elements, such as a copper plate, light filter, and/or layer of piezoelectric material that reacts to being bent. The layer of piezoelectric material may function as a vibroacoustic sensor.

In certain embodiments, the sensors facing the user's body include a vibroacoustic sensor, a PPG/SpO2 sensor, and an electric potential sensor. The sensors facing away from the user's body include a pressure sensor, a temperature sensor, a humidity sensor, a light sensor, and an inertial measurement unit (IMU). In other embodiments, any other combination of sensors for detecting physiological and/or environmental signals may be used in the foldable sensor device 200. The types of sensors that can be used with the present technology is not particularly limited, and certain example sensors are described herein.

The substrate 205, join member 212, substrate 213, join member 214, and/or substrate 215 may include other electronic components, such as communication components including an antennae, power sources including a battery, storage devices including flash memory which may be removable, processors, a Universal Serial Bus (USB) port or other data transmission port, shielding components, grounding components and/or a signal amplifying component. One or more batteries may be included in the foldable sensor device 200. The batteries may be attached to the substrate 205, the substrate 213 and/or substrate 215. When the foldable sensor device 200 is folded, the batteries may be sandwiched between the substrate 205 and substrate 215. The batteries provide power to the sensors and/or other electronic components of the foldable sensor device 200. The antenna may be incorporated in the join members 212 and/or 214.

The foldable sensor device 200 may include a storage unit for storing data collected by the sensors. The storage unit may be communicatively coupled to the sensors to receive the data captured by the sensors. The storage unit may be accessed by a processor of the foldable sensor device 200. The data stored on the storage unit may be accessed via the USB port of the foldable sensor device 200 and/or via a wireless communication protocol, such as Wi-Fi or Bluetooth. The storage device may be removable, such as a removable flash memory device.

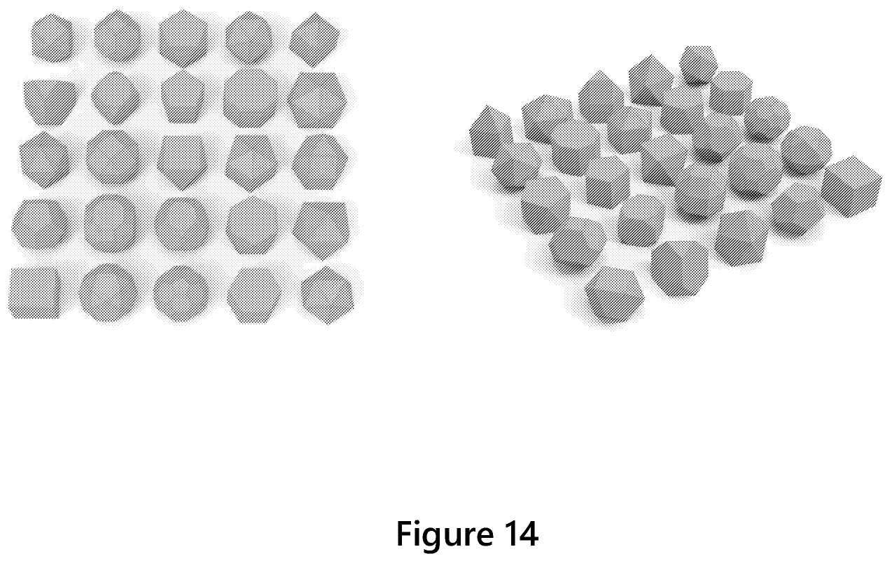

The foldable sensor device 200 may have various shapes beyond the illustrated embodiment. For example the foldable sensor device 200 may be derived from a polyhedron which is flattened (unfolded configuration) then folded (folded configuration). This is described later in relation to FIG. 14. In one example, the polyhedron may have six faces which when flattened and then folded would create six substrate layers. The arrangement of the sensors on the faces of the substrates may differ from that as illustrated.

### Modular Substrates

The substrates 205, 213, and/or 215 may be removable from the foldable sensor device 200. The substrates 205, 213, and/or 215 may be replaceable. For example, the substrate 205 may be detached from the join member 212. Then, another substrate may be attached to the join member 212. The replacement may have a same size and/or shape as the substrate 205. By removing and/or replacing substrates, the foldable sensor device 200 may be configured for various different uses. The substrates 205, 213, and/or 215 may be selected based on a desired use of the multi-layer sensor device. For example, if the foldable sensor device 200 is not intended to measure physiological data of a human, the selected substrates 205, 213, and/or 215 may be substrates that contain environmental sensors but not physiological sensors. In another example, if the foldable sensor device 200 is being used in a setting where it will not be recharged frequently, one of the selected substrates 205, 213, and/or 215 might contain batteries rather than sensors. In this manner the foldable sensor device 200 can be configured for a particular purpose by selecting substrates that correspond to the purpose, and attaching those substrates to join members.

In this respect, in certain embodiments, the foldable sensor device 200 may comprise one or more substrates which have been pre-prepared with the sensors housed thereon. The foldable sensor device 200 is thus made by selecting the desired combination of substrates and attaching them to one another by one or more join members. In these embodiments, the manner of how the substrates and/or the join members are attached to one another is not limited. For example, the attachment may be by means of mechanical fasteners, or by welding, adhesive, or in any other manner.

### Join Member

In certain embodiments, a join member connecting two substrates may have a twisted configuration when the foldable sensor device is in an unfolded configuration. This may permit ease of manufacture during connecting sensors to one side of one substrate and the other side of the other substrate. The join member may be made of a piezoelectric material which can be caused to change form to untwist and thereby change an orientation of the substrates to one another.

Foldable Sensor Device in a folded configuration

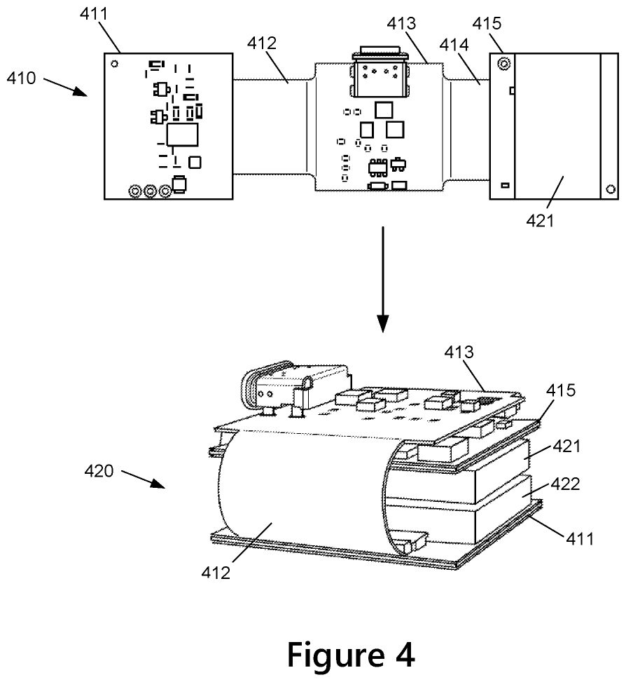

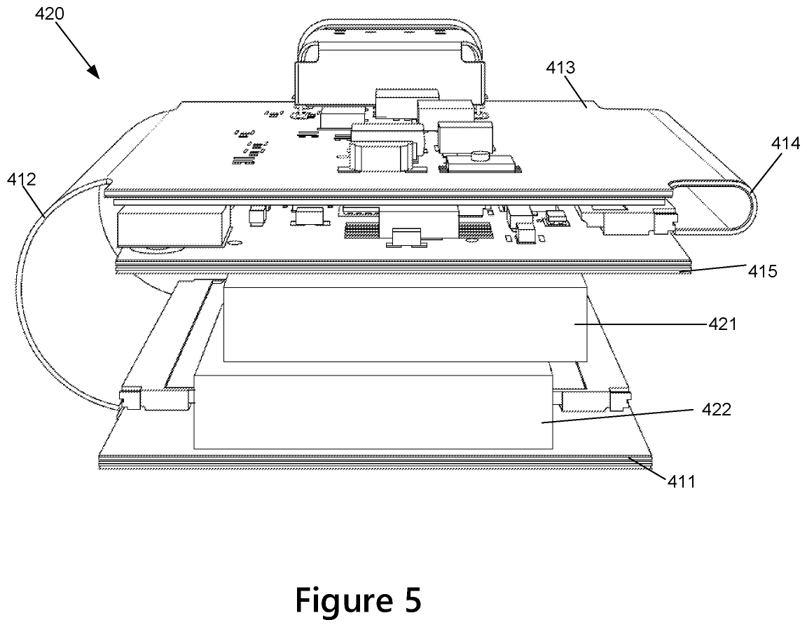

FIGS. 4 and 5 illustrate assembly of a foldable sensor device in a folded configuration 420 (also referred to as folded sensor device 420), from a foldable sensor device in an unfolded configuration 410 (also referred to as unfolded sensor device 410), in accordance with various embodiments of the present technology.

Similar to the foldable sensor device 200 of FIGS. 2 and 3, the unfolded sensor device 410 includes a substrate 411, a substrate 413, a substrate 415, and join members 412, and 414. In the unfolded configuration, the substrate 411, substrate 415, and join members 412, 413, 414 are co-planar. In the view illustrated in FIG. 4, a first side of the substrate 411 supports sensors and/or electronic components. Similarly, a first side of the join member 413 supports sensors and/or electronic components, including a USB port. A first side of the substrate 415 supports a battery 421. A second side of the substrate 411 includes a battery 422, which cannot be seen from the illustrated first side view.

As for the join members 212, 214 of FIGS. 2 and 3, the join members 412 and 414 are configured to permit the unfolded device 410 to be formed into the folded sensor device 420. The join members 412 and/or 414 may permit folding by virtue of a flexible property of the join members. Alternatively, the join members 412 and/or 414 may be formed of a more rigid material and have a hinge-like structure. In the folded configuration, the substrate 411 may form a first layer that faces the skin of the user. The sensors illustrated on the surface of the substrate 411 may be on the bottom surface of the folded sensor device 420 and/or may be positioned to face the user's skin in use. The substrate 415 may form a second layer of the folded device 420. Between the two substrates 411 and 415 are the two batteries 421 and 422.

The join member 413 forms a top surface of the folded sensor device 420. The top surface may face away from the user's body in use. The join member 413 and/or substrate 415 may contain environmental sensors. The environmental sensors may obtain and/or record data about the environment surrounding the user.

FIG. 5 illustrates another view of the folded sensor device 420. The folded sensor device 420 may remain in the folded configuration when it is placed in an enclosure. The enclosure may maintain the folded sensor device 420 in the folded configuration. The enclosure may be a wearable device and/or a device that is not intended to be worn by a user. For example the enclosure may be an enclosure of a device that can be attached to a structure, such as a tower.

Alternatively, the folded sensor device 420 may retain the folded configuration by means of a retaining member (not shown), such as a clasp, extending between an upper and lower substrate.

### Sensor Configuration

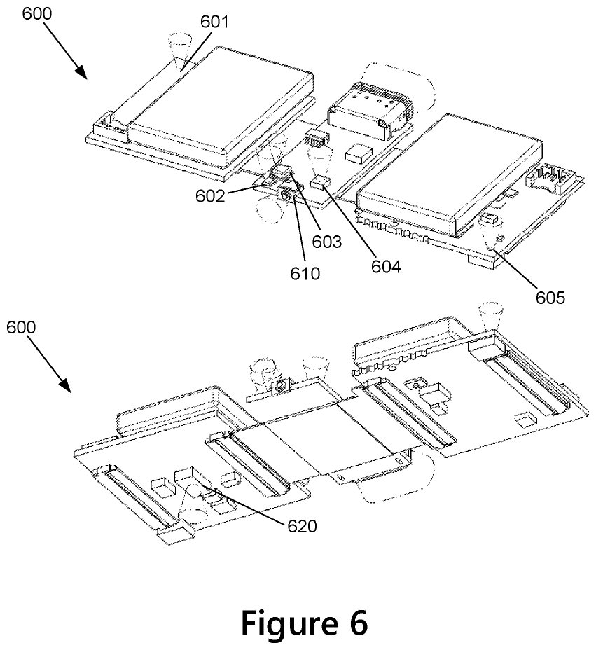

FIG. 6 illustrates a foldable sensor device 600 which is another embodiment of the foldable sensor devices 200, 410 in the unfolded configuration, in accordance with various embodiments of the present technology. As for the foldable sensor devices 200, 410, the foldable sensor device 600 includes a plurality of substrates for supporting one or more sensors and which can be folded relative to each other to form a stacked configuration of the substrates.

FIG. 6 illustrates a top view and a bottom view of the sensor device 600 (upper and lower images, respectively). As can be seen in FIG. 6, sensors 601, 602, 603, 604, and 605 capture data from a first direction. Sensor 620 captures data from a second direction, which is opposite of the first direction. The sensor 610 captures data from a third direction, which is perpendicular to the first and second directions.

### Form Factors

Any of the foldable sensor devices of the present technology may be provided with an enclosure for supporting the foldable sensor device and enabling it to be attached or positioned close to the body of the subject.

The enclosure may have any suitable form factor, such as, without limitation a wearable configuration, for example: a strap, a band aid, a patch (e.g. drug delivery patch with or without medication), a watch, a bandage, an item of jewelry, a head piece (e.g. helmet such as a military, safety/protective or sports helmet, hat, headband), an eye piece (e.g. goggles, spectacles, monocles, safety spectacles, sun glasses, visors (e.g. for sports, safety, military) or other eye wear), an ear piece (e.g. hearing aid, earphones, earbuds, headphones, ear covering shields), a mouth piece (e.g. mouthguards, self-contained breathing apparatus (SCBA)), a device for the face (e.g. masks or visors for surgical, sport, or other protective use; ski masks; swimming goggles), devices for the wrist or arm (e.g. smart watches, watches, connected bracelets, wearable activity trackers), a collar, an item of clothing (e.g. for protection against accidents and against fire; body armor; footwear such as boots, shoes, trainers; vests, hijabs, niqabs, abayas, mittens, gloves; uniforms such as for sport or military, belts), a support (e.g. knee, wrist, elbow, hip and shoulder pads for athletic or medical use; shin guards, bedding), devices for invasive use (e.g. a cervical collar), collars for animals, jewelry, blankets, pillows, bedding or cushions.

In other embodiments, the enclosure may have a configuration other than a wearable configuration, such as for example a picture frame, a mirror, a piece of furniture, a portion of a building, wall, ceiling, structural building parts, machinery.

In any type of enclosure, a position of a given sensor in the enclosure may be such that a proximity to a target body part is optimized. For example, for non-contact applications, a given sensor for detecting electrical activity related to the brain may be included in a forehead facing side of a headband.

### Band-Aid Form Factor

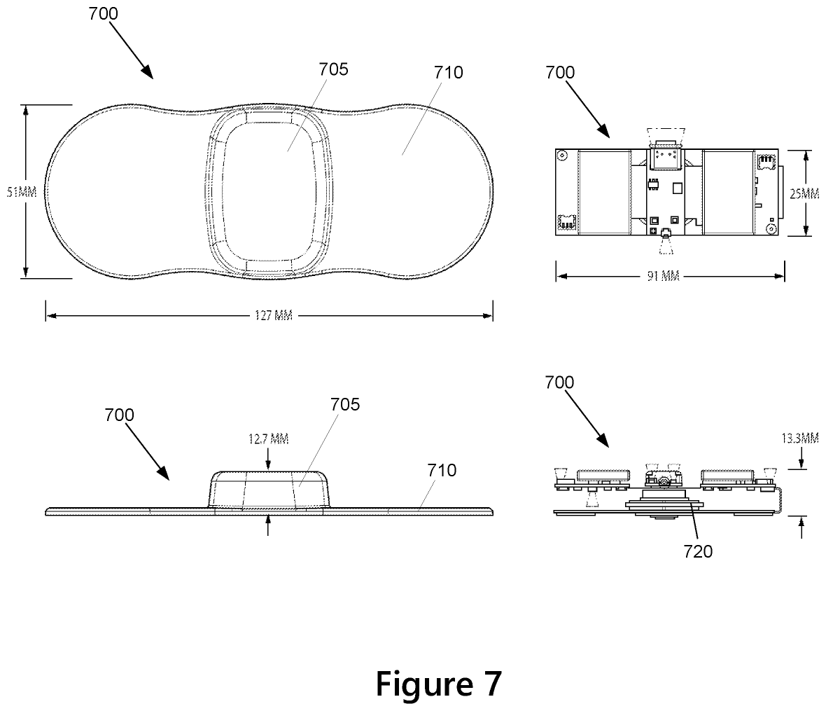

FIG. 7 illustrates an embodiment of a wearable sensor device 700 comprising a foldable sensor device 705 in the folded configuration supported by an enclosure 710, such as by embedding. The enclosure 710 comprises a band-aid-like structure. The enclosure 710 may be placed against the user's skin.

In certain embodiments, at least a portion of the enclosure 710 comprises a breathable material and includes an adhesive for attaching it to the skin of the user. At least a portion of the enclosure 710 is flexible and can conform to a shape of the body part of the user (such as a neck, an arm, a leg, a torso). In certain embodiments, at least a portion of the enclosure 710 is waterproof or water resistant.

As for the embodiments described with reference to FIGS. 2-6, the foldable sensor device 705 comprises a plurality of substrates for supporting sensors and/or other electronic components and which can be folded relative to each other so that the substrates are stacked.

More specifically, the sensor device 705 includes a vibroacoustic sensor 720. The vibroacoustic sensor 720 is positioned between two layers of the illustrated multi-layer sensor device 700.

In certain embodiments, the wearable device 700 is less than about 15 mm high and is therefore discreet when worn by the user. In other embodiments, the wearable device 700 is less than about 35 mm high, less than about 30 mm high, less than about 25 mm high, or less than about 20 mm high. The dimensions included in FIG. 7 are exemplary, and it should be understood that other dimensions may be used.

### Centered Configuration

The vibroacoustic sensor 720 (and for that matter any other sensor), may be positioned centrally in the wearable device 700. Other sensors, such as electric potential sensors, may be placed towards the edges of the wearable 700. Positioning sensors on the left side, right side, and/or center of the wearable 700, may allow for a more differential signal to be captured by the sensors, because of the increased distance between the sensors. This configuration may be preferable for placement over various body locations, such as over the user's heart. This configuration may be laterally the most compact of the configurations illustrated in FIGS. 7-10.

### Off-Center Configuration

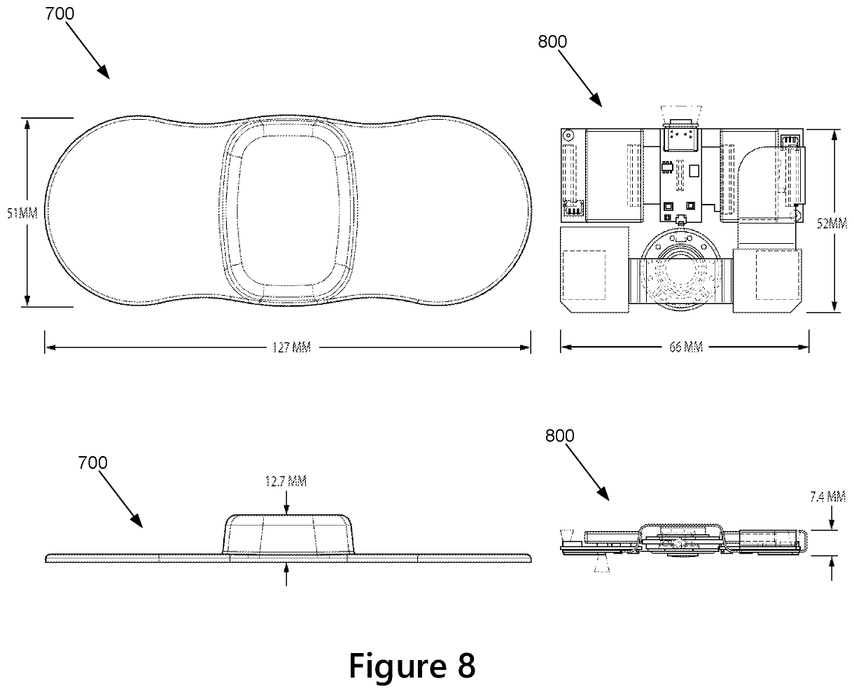

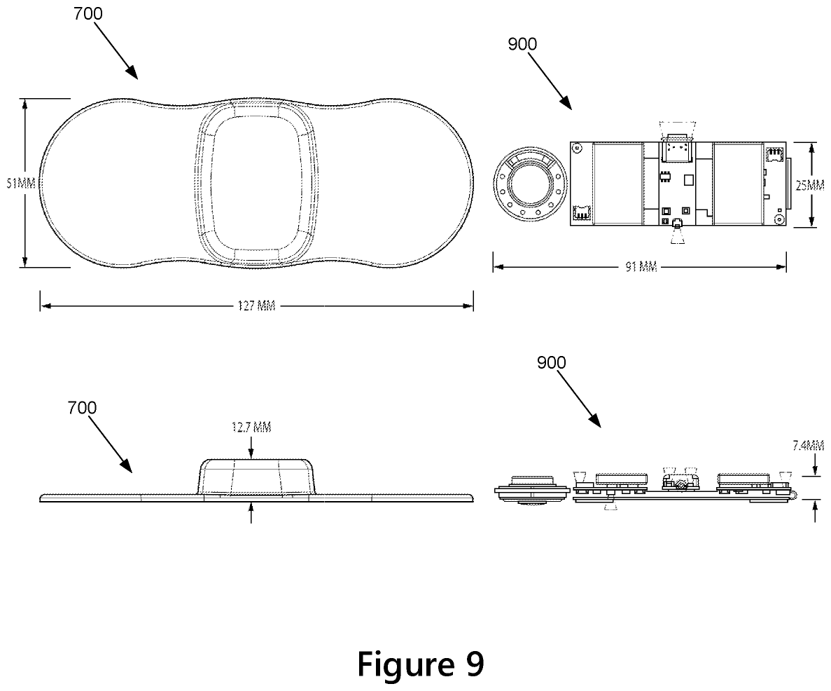

The wearable device 700 of FIGS. 8 and 9 differ from the embodiment of FIG. 7 in that respective unfolded configurations 800, 900 have a side-by-side configuration in accordance with various embodiments of the present technology. The configurations 800 and 900 may have a lower thickness than the configuration 700, and may be preferable for use in enclosures having a smaller thickness, or in any other instance where a wearable device having a relatively lower thickness is preferable. Because of the lower thickness, the configurations 700 and/or 800 may be more easily concealed and/or more comfortable for a user to wear.

### In-Line Configuration

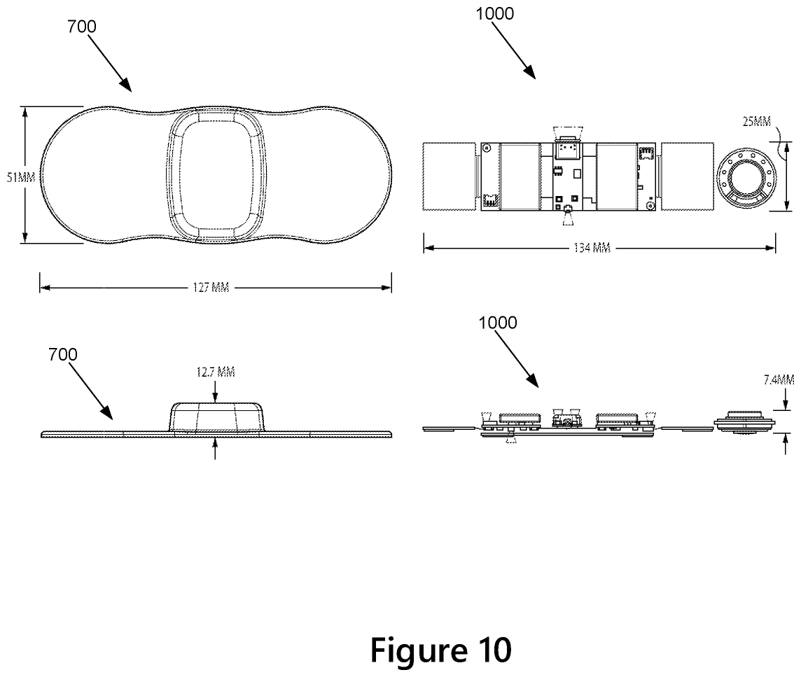

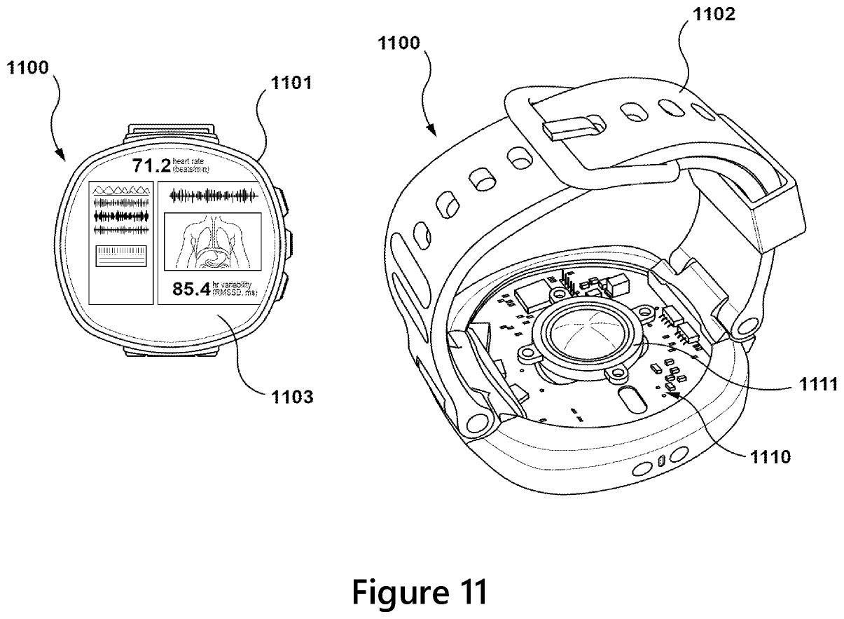

The wearable device 700 of FIG. 10 differs from the embodiment of FIG. 7 in that unfolded configuration 1000 has an in-line configuration in accordance with various embodiments of the present technology. Similarly to the configurations 800 and 900, the configuration 1000 may have a lower thickness than the configuration 700. This configuration may be preferable for integration in a wearable device such as the watch 1100, which is described in further detail below and in FIG. 11. In the configuration 1000, the vibroacoustic sensor is positioned to the side of other sensors. In the configuration 1000, the vibroacoustic sensor might not positioned between electric potentials sensors, beamforming MEMS microphones, and/or other sensors.

### Watch Form Factor

As discussed above, embodiments of the foldable sensor device of the present technology may include an enclosure with any type of form factor. FIG. 11 illustrates a watch 1100 with an integrated foldable sensor device 1110 in accordance with various embodiments of the present technology. In other words, the enclosure comprises a watch-like structure. The watch 1100 may be worn by a user. The watch 1100 may include an enclosure 1101 and band 1102 attached to the enclosure and fastenable around a wrist of the user. The enclosure 1101 may include a display 1103. The enclosure 1101 may contain the foldable sensor device 1110. The foldable sensor device 1110 is in a folded configuration and may comprise any of the structures described or illustrated herein such as the foldable sensor devices of FIGS. 2-10. A portion of the foldable sensor device 1110 may face the skin of the user and/or directly or indirectly contact the skin of the user. The foldable sensor device 1110 may include a vibroacoustic sensor 1111. The vibroacoustic sensor 1111 may be positioned to face the skin of the user and/or contact the skin of the user. Other sensors may include one or more of: a GPS, an IMU, a humidity sensor, a barometric pressure sensor, ambient temperature, core body temperature, MEMS microphone, ambient light, ionization radiation detector, radiofrequency and radiation sensor, pulse rate, respiratory rate, body temperature, blood pressure and peripheral capillary oxygen saturation sensor, electric potential sensor (e.g. ECG and AC bioimpedance, skin conductance, galvanic skin response, electrodermal response, electrodermal activity), ballistocardiography sensor.

The watch 1100 may comprise ultralow power consumption electronics with personalizable 3-D printed casings which comprise Titanium/Titanium Dioxide dielectric film, silicon dioxide dielectric film, or gold standard Ag/AgCl electrodes to provide measurements of the raw ECG waveform, heart rate, and meanNN and SDNN heart rate variability parameters. The battery life may comprise more than a month of battery life for a weight of less than 100 g.

The display 1103 may display data collected by sensors of the foldable sensor device 1110, such as physiological data corresponding to the user and/or environmental data corresponding to the environment surrounding the user. The watch 1100 may store and/or transmit the collected sensor data. The watch 1100 may include a wireless networking device to transmit the data via a wireless interface, such as via Wi-Fi and/or Bluetooth.

The watch 1100 may be configured to have a number of operating modes including one or more of: a continuous data collection mode; an intermittent data collection mode at fixed time-intervals; and a threshold-triggered data collection mode. A given sensor in the watch 1100 may have a different collection mode than another given sensor. For example, the GPS and pressure sensors may operate under the continuous data collection mode whereas the vibroacoustic sensor 1111 may operate under the threshold-triggered data collection mode.

### Other Unfolded Configurations

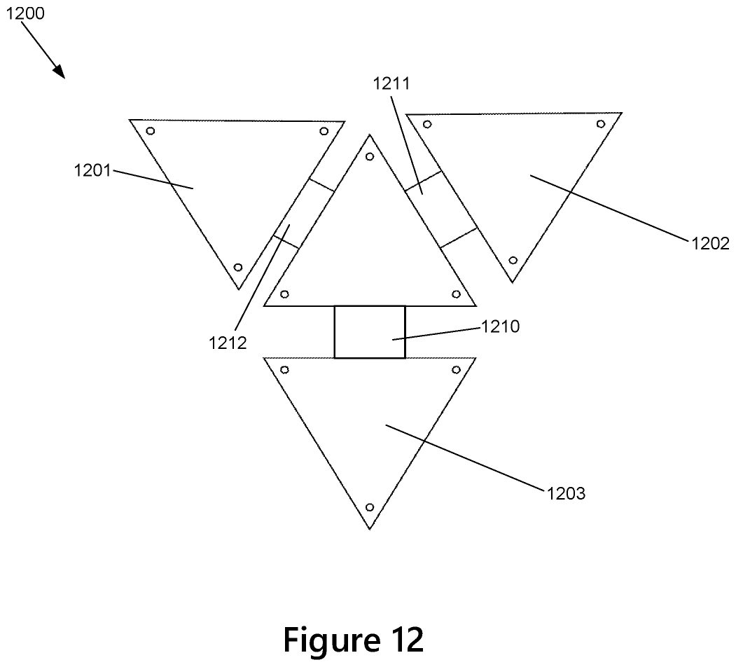

FIG. 12 illustrates an unfolded configuration of a foldable sensor device 1200 with a central substrate 1204. The multi-layer sensor device 1200 includes the central substrate 1204 and three other substrates 1201, 1202, and 1203. Join member 1212 connects the substrate 1201 and the central substrate 1204. Join member 1211 connects the substrate 1202 and the central substrate 1204. Join member 1210 connects the substrate 1203 and the central substrate 1204.

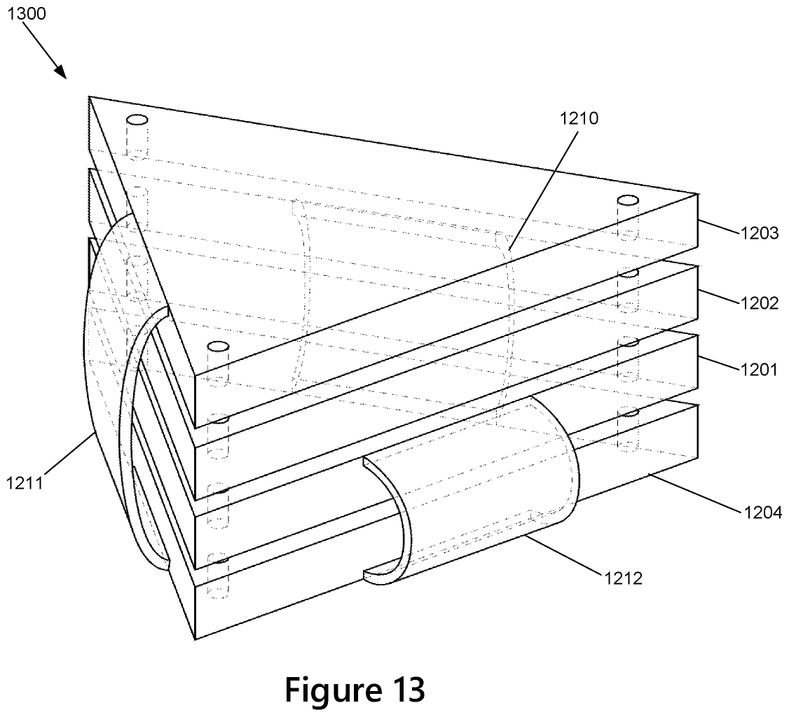

A length of the join member connecting a substrate to the central substrate 1204 may define how the multi-layer sensor device 1200 is folded. The join layer 1212 has the shortest length of the join members 1210-12 illustrated in FIG. 12. Because the join layer 1212 has the shortest length, the substrate 1201 may be folded first, on top of the central substrate 1204. As illustrated in FIG. 13, the substrate 1201 may be adjacent to the central substrate 1204 in the folded configuration. The join member 1211 is the second-shortest join member of the multi-layer sensor device 1200. Because the join member 1211 is the second-shortest join member, the substrate 1202 may be folded next and placed on top of the substrate 1201. Finally, the join member 1210 is the longest join member of the multi-layer sensor device. Because the join member 1210 is the longest join member, the substrate 1203 may be folded last and placed on top of the substrate 1202.

Like the substrates described above, the central substrate 1204 and substrates 1201-03 may contain sensors, batteries, and/or any other electronic components.

FIG. 13 illustrates a folded configuration of the foldable sensor device 1300 with the central substrate 1204. In the folded configuration, the central substrate 1204 forms a first layer, the substrate 1201 forms a second layer, the substrate 1202 forms a third layer, and the substrate 1203 forms a fourth layer.

In this respect, in the folded configuration, the foldable sensor device 1300 may be defined as having a plurality of substrate layers for supporting one or more sensors and wherein each substrate layer is connected to at least one other substrate layer by a join member.

### Substrate Selection

In certain embodiments, the number of substrates and the arrangement of substrates of the foldable sensor devices may be determined from polyhedral shapes. More specifically, a 3D polyhedral shape, such as those illustrated in FIG. 14, may be opened up and converted to a 2D flat form by “cutting” along one or more edges. Each face of the 2D flat polyhedral shape may thus define a substrate of the foldable sensor device, with a join member between at least some of the faces. It will be appreciated that depending on how the 3D shape is opened up to the 2D flat form will affect the folding pattern of the subsequent foldable sensor device.

### Methods

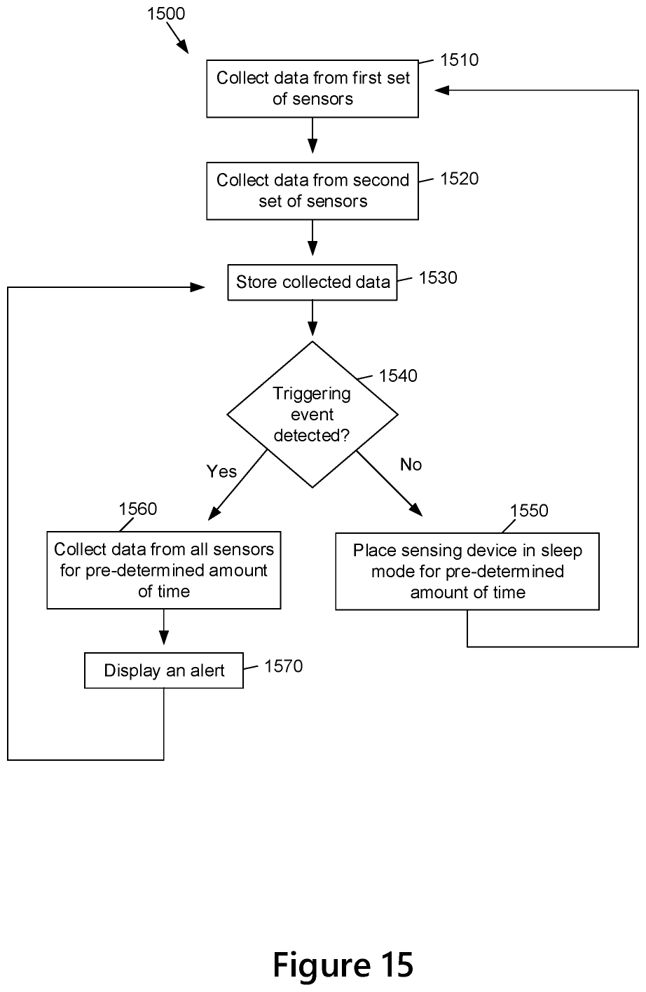

FIG. 15 is a flow diagram of a method 1500 for collecting data in accordance with various embodiments of the present technology. In one or more aspects, the method 1500 or one or more steps thereof may be performed by a computing system, such as the computing environment 100. All or a portion of the steps may be executed by any of the foldable sensor devices described herein, such as by a processor and memory of the foldable sensor device. The method 1500 or one or more steps thereof may be embodied in computer-executable instructions that are stored in a computer-readable medium, such as a non-transitory mass storage device, loaded into memory and executed by a CPU. Some steps or portions of steps in the flow diagram may be omitted, changed in order, and/or executed in parallel.

As discussed above, a foldable sensor device may include multiple sensors, including a first set of sensors that measure data relating to the user and a second set of sensors measuring data relating to the environment surrounding the user. In order to conserve power, the first and/or second sets of sensors of the foldable sensor device may be deactivated for certain time periods. In order to collect more relevant data, the sensors of the foldable sensor device may be activated upon detection of a triggering event. In certain embodiments, the sensors within each of the first set and second set of sensors may be controlled individually. Rules determining when and how data is collected may be determined by a data collection protocol. The data collection protocol may take into consideration balancing battery life with collection of pertinent data or storage of pertinent data. For example, environmental data may be continuously monitored, but vibroacoustic data of the user may be collected when triggered based on a detected environmental event.

At step 1510, data may be collected from the first set of sensors. At step 1520, data may be collected from the second set of sensors. Although two sets of sensors are described, data may be collected from any number of sensors, and those sensors may be divided into any number of sets. The data may be collected simultaneously from both sets of sensors, in which case steps 1510 and 1520 may be executed in parallel. The first set of sensors may be sensors facing the user's body. As described above, these sensors may collect physiological data and/or other data related to the user. The second set of sensors may be sensors facing away from the user's body. These sensors may collect data relating to the environment surrounding the user.

Data may be collected from the first set of sensors and second set of sensors at different intervals. Data may be collected from the first set of sensors more frequently than data collected from the second set of sensors. In that case, data corresponding to the user may be collected at a higher frequency than the data corresponding to the environment surrounding the user. A frequency for collecting data may be assigned to each sensor and/or set of sensors. Alternatively, data may be collected from the first set of sensors and second set of sensors at same intervals.

At step 1530, the collected data may be stored. The data may be stored locally on the foldable sensing device, such as in a flash memory of the foldable sensing device. The data may be stored in a memory device that is in communication with the foldable sensing device. For example, the watch illustrated in FIG. 11 may contain a storage device separate from the foldable sensing device. The collected data may be stored in the storage device of the watch. The data may be transmitted to an external device and stored by the external device. For example the data may be transmitted to a smartphone and/or a cloud service. The data may be encrypted prior to storage and/or transmission.

At step 1540, a determination may be made as to whether a triggering event was detected in the data collected at steps 1510 and/or 1520. Data collected by the first and/or second set of sensors may be continually monitored to determine whether a triggering event has occurred. The triggering events may be predefined events. The triggering events may be defined globally for all sensors, for a subset of sensors, and/or for each individual sensor. Each pre-defined trigger may include an indication as to which sensors the trigger should be applied to. Triggers may include data from an individual sensor and/or a combination of multiple sensors.

A predefined threshold minimum and/or maximum value may be defined as a triggering event for a sensor and/or set of sensors. If the measured data exceeds the maximum value and/or is lower than the minimum value, the triggering event may be detected and the method 1500 may proceed to step 1560. The triggering event may be defined as a change above a pre-defined threshold in the measured data. For example if the measured data from a sensor changes more than twenty percent over a time period of ten seconds, a triggering event may be recorded and the method 1500 may proceed to step 1560.

If no triggering event is detected at step 1540, the method 1500 may continue to step 1550 and place the sensing device in a sleep mode for a pre-determined amount of time. In one embodiment a single pre-determined amount of time may be applied to all sensors of the multi-layer sensing device. In another embodiment a different pre-determined amount of time may be applied to the sets of sensors of the multi-layer sensing device. In yet another embodiment a pre-determined amount of time may be defined for each sensor and/or type of sensor. As discussed above, the frequency that data is recorded may be different for each set of sensors and/or individual sensor. After the pre-determined amount of time has passed, the method 1500 may proceed to steps 1510 and 1520 and data may be collected from the sensors.

If a triggering event was detected at step 1540, the method 1500 may proceed to step 1560. At step 1560 data may be collected from all sensors at a higher frequency for a pre-determined amount of time. The amount of time may be determined based on the triggering event that occurred. If, after the pre-determined amount of time, the conditions that caused the triggering event continue, the data may continue to be collected at the higher frequency. In one embodiment, all triggering events may cause a same set of actions to occur at step 1560. In another embodiment, each triggering event may define a set of actions to be performed at step 1560. For example the triggering event may define the pre-determined amount of time to collect data, the sensors to collect data from during the pre-determined amount of time, and/or the frequency at which data is to be collected from individual sensors during the pre-determined amount of time.

At step 1570 an alert may be displayed. The alert may indicate that the triggering event has occurred and/or may provide a warning to the user. For example if a triggering event in the environment surrounding the user has been detected an alert may warn the user that they are in an unsafe environment. After displaying the alert, the method 1500 may proceed to step 1530 and the data collected during the triggering event may be stored. The method 1500 may then proceed to step 1540 to determine whether the triggering event has continued.

### Manufacturing the Multi-Layer Sensor Device

Broadly, a method of manufacturing a foldable sensor device comprises providing a plurality of substrates having associated therewith one or more sensors and/or other electronic components and folding the plurality of substrates relative to each other to form a stacked multi-layer substrate configuration.

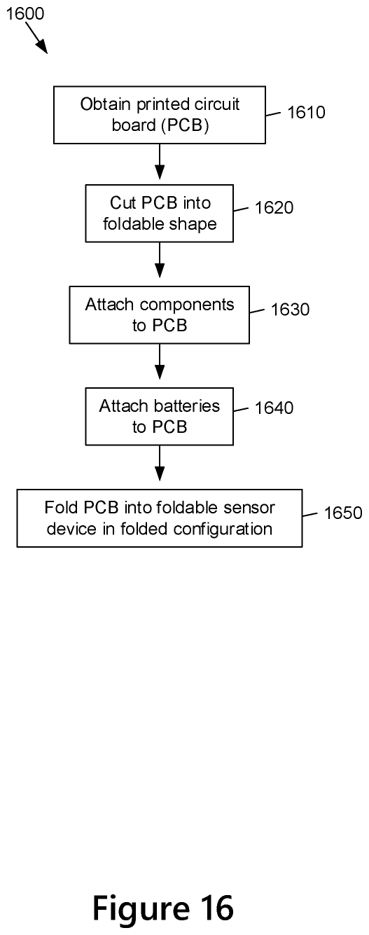

For foldable sensor devices in which the substrates and the join members have a common material, the step of providing the plurality of substrates may comprise providing a one-piece material made of a join member material and having substrate portions and join member portions, and attaching a stiffener material to the substrate portions so that the substrate portions are more rigid than the join member portions. This will be described further with reference to FIG. 16.

Alternatively, the step of providing a plurality of substrates may comprise obtaining individual substrates and joining them together using join members. The individual substrates may already have sensors or other electronic components attached thereto or otherwise formed therein. Indeed, in certain embodiments, the individual substrates may be selected from a plurality of different substrate types, each substrate type having a given suite of sensors suitable for a given use.

FIG. 16 is a flow diagram of a method 1600 for manufacturing a foldable sensor device in accordance with various embodiments of the present technology. FIG. 16 describes an exemplary method of manufacturing the foldable sensor device, but any other suitable method may be used. The method 1600 may be modified depending on the characteristics of the desired foldable sensor device, including based on the size, shape, number of layers, sensor configuration, and/or any other characteristics of the foldable sensor device. The method 1600 may be used to manufacture any of the foldable sensor devices described herein.

At step 1610 a printed circuit board (PCB) may be obtained or formed. The PCB may be printed and/or otherwise manufactured using any suitable PCB fabrication technique. The PCB fabrication process may include various actions, including etching, drilling, plating, coating, printing, and/or any other actions for fabricating a PCB.

At step 1620 the PCB may be cut into a shape suitable for forming a foldable sensor device and including substrate portions and at least one join member portion. FIGS. 2-4, 6-10, and 12 illustrate various exemplary shapes that the PCB may be cut into. The shape may be determined based on an amount of desired layers of the folded multi-layer sensor device and/or a desired size of the folded multi-layer sensor device. For example, the at least one join member portion may be necked or thinned compared to the substrate portion to facilitate bending. Optionally, a stiffener material may be added to one or more of the substrate portions.

At step 1630 components may be attached to the PCB. The components may include sensors and/or other electronic components. The components may be placed on one or both sides of the PCB, such as a top surface of the PCB and/or a bottom surface of the PCB. Any number of components may be attached to the PCB. The components may be co-planar. It will be appreciated that attaching sensors to one side of the PCB has advantages in terms of an ease and simplicity of manufacture.

At step 1640 batteries may be attached to the PCB. The batteries may be attached directly to the PCB and/or attached to a substrate on the PCB. The batteries may be placed on a top surface of the PCB and/or the bottom surface of the PCB. Any number of batteries may be attached to the PCB.

At step 1650 the PCB may be folded to form a foldable sensor device in the folded configuration. For example the PCB may be folded to form the foldable sensor devices illustrated in FIGS. 4, 5, 7-10, and 13. The foldable-layer sensor device may then be placed in an enclosure, such as a wearable enclosure.

### Predicting or Monitoring a Condition of a User

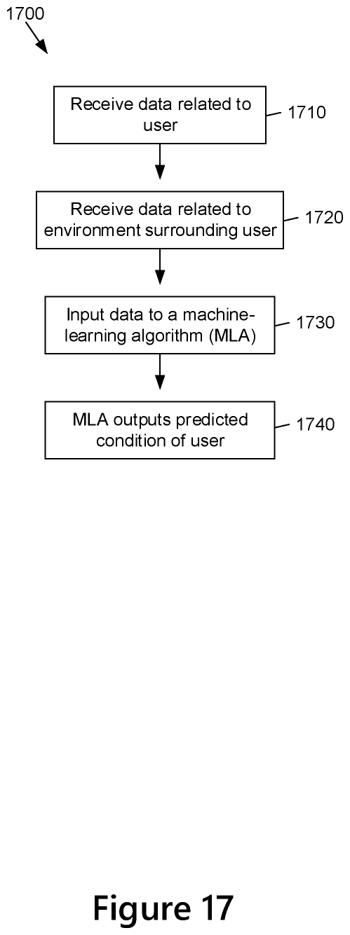

FIG. 17 is a flow diagram of a method 1700 for predicting or monitoring a condition of a user in accordance with various embodiments of the present technology. In one or more aspects, the method 1700 or one or more steps thereof may be performed by a computing system, such as the computing environment 100. All or a portion of the steps may be executed by any of the foldable sensor devices described herein, such as by a processor and memory of the foldable sensor device and/or by a computing environment 100 in communication with the foldable sensor device. The method 1700 or one or more steps thereof may be embodied in computer-executable instructions that are stored in a computer-readable medium, such as a non-transitory mass storage device, loaded into memory and executed by a CPU. Some steps or portions of steps in the flow diagram may be omitted, changed in order, and/or executed in parallel.

At step 1710 data related to the user may be received. The data related to the user may be collected by sensors on a foldable sensor device which is in a folded configuration and are facing the user.

At step 1720 data related to the environment surrounding the user may be received. The data related to the environment surrounding the user may be collected by sensors facing away from the user on the foldable sensor device in the folded configuration. The data related to the environment surrounding the user may be collected by one or more foldable sensor devices that are proximal to the user, such as enclosed in devices worn by the user, and/or one or more foldable sensor devices that are located in the environment surrounding the user. The foldable sensor devices in the environment surrounding the user may be attached to structures, such as a building or a tower. The data related to the environment surrounding the user may be collected from wearable devices worn by other individuals in the environment. The data from the other individuals may be data collected by sensors facing the individuals and/or sensors facing away from the other individuals. In other words, the data from the other individuals may include data relating to the other individuals and/or data relating to the environment surrounding the other individuals.

In either or both of steps 1710 and 1720 the data may comprise raw signal data captured by the sensors of the foldable sensor device, or be a filtered and/or amplified data set.

At step 1730 the data received at steps 1710 and 1720 may be input to a machine-learning algorithm (MLA). The MLA may have been trained to predict, based on data collected by a foldable sensor device, whether an individual is subject to a condition, such as a medical condition. The MLA may have been trained to predict other information, such as whether the individual was attacked, for example by a directed energy attack. In order to make the prediction, the MLA may analyze features of the data corresponding to the user and/or the data corresponding to the environment surrounding the user.

The data received at steps 1710 and/or 1720 may be input to an MLA trained to detect the use of a directed energy weapon. The MLA may analyze features of data collected by devices worn by the user and/or devices in the environment surrounding the user.

At step 1740 the MLA may output the predicted medical condition of the user, whether a directed energy weapon was used, and/or any other predictions that the MLA has made based on the data. A user interface describing the predictions may be displayed to the user or to a third party. In this respect, the processor may cause an alert to be sent to an electronic device such as a smart phone of the third party.

By condition is meant one or more of a medical condition, a cognitive load state, an alertness state and an activity of the user or a state of mind of the user.

Example, non-limiting, conditions are set out below:

### Depression

The condition being monitored or predicted by embodiments of the present technology may comprise depression. This condition may be predicted or monitored based on a response time measurement of the user. Depression is often accompanied by less efficient cognitive function, as indicated by slower speed of information processing. Slower processing speed, as indexed by longer simple choice reaction time (SCRT) or choice reaction time (CRT), can be associated with an increased risk of psychological distress.

Behavior and/or Activity

The condition being monitored or predicted by embodiments of the present technology may comprise a behavior or an activity of the user. For example, a human activity recognition (HAR) system may be incorporated for monitoring activity patterns. In case the processor detects a change in behavior or a critical event (e.g. a fall), an alert may be sent to the user or a third party. This can enable users to have a more independent life.

By activity is also meant the presence of a person in a monitored area around the foldable sensor device. The activity of the person in proximity to the non-contacting foldable sensor device may include a moving body part such as torso, head, or limb of a person.

Recovery after Exertion

The condition being monitored or predicted by embodiments of the present technology may comprise recovery after exertion as an indicator of a fitness level of the user, or post-operative healing for example. In this respect, the sensors may be configured to obtain data relating to objective sleep-wake cycles as they relate to stress and exertion recovery. The devices, methods and systems of the present technology may make inferences about recovery between exertion periods. These include:
Heart Rate Variability (HRV) is a measure of the difference in the amount of time between successive heart beats, and can also be captured during the last period of Slow Wave Sleep each night. HRV is an indicator of the health of the autonomic nervous system, and a trending increase in HRV leads to a stronger recovery.
Resting Heart Rate (RHR) is a measure of the number of heart beat when at complete rest. This can also be captured during the last period of Slow Wave Sleep each night. Lower Resting Heart Rates over time are an indication of improved fitness and Recovery.
Respiratory Rate is the number of breaths taken a minute on average over the course of wake and/or sleep cycles. Respiratory rates vary according to the level of exertion. Night over night, Respiratory Rate shouldn't fluctuate much. Respiratory Rate can impact Recovery if breath rate increases significantly above normal levels.

Sleep is when your body recovers. Getting more restful sleep each night can improve recovery the following day, as well as over time. However, getting 8 hours of sleep will not guarantee good recovery, and getting 4 hours of sleep will not guarantee bad recovery. Other factors in recovery are: Fitness level, Health, Behavior, Stress, Diet, Hydration, Recent Strain and Drug use including alcohol and tobacco.

### Objective Pain

The condition being monitored or predicted by embodiments of the present technology may comprise a pain being felt by the user. Pain scales are useful for the assessment of pain and also for monitoring the effectiveness of treatment. It is therefore important that pain scales are simple and efficient. Subjective pain scores are currently used for acute pain management. The assessment of pain by the patient as well as the caregiver can be influenced by a variety of factors. The numeric rating scale (NRS) is widely used due to its easy application. The NRS requires abstract thinking by a patient to assign a score to correctly reflect analgesic needs, and its interpretation is subject to bias. Pain is a subjective feeling, and the self-assessment of pain by the patient and evaluation by the observer can be influenced by a variety of factors, including but not limited to socio-economic status, beliefs, and psychological status. The present technology uses contextualized multi-parameter autonomic data combined with Structural ML to provide an objective pain scoring solution that can eliminate the subjective component from the assessment of pain, as indicated by the excessive use of opioids as well as under treatment of pain can have adverse effects on enhanced post-surgical recovery.

### Objective Mood

The condition being monitored or predicted by embodiments of the present technology may comprise a mood or a mental health disorder of the user. Mental health disorders have a devastating impact on an individual's health and happiness. Worldwide, it is estimated that four of the ten leading causes of disability for persons aged five and older are mental disorders. Among developed nations, major depression is the leading cause of disability. By some estimates, mental health problems affect one in four citizens at some point during their lives. As opposed to many other illnesses, mental ill health often affects people of working age, causing significant losses and burdens to the economic system, as well as the social, educational, and justice systems. The economic burden of these illnesses exceeds $300 billion in the US alone. Despite these facts, the societal and self-stigma surrounding mental health disorders have remained pervasive, and the assessment, diagnosis, and management of these starkly contrasts with the numerous technological innovations in other fields of healthcare.

Previous research has also indicated that identifying reliable indicators of depression is non-trivial. Although the assessment of behavior is a central component of mental health practice, it is severely constrained by individual subjective observation and lack of any real-time naturalistic measurements. Symptoms of depression can vary greatly both within and between individuals. Moreover, people naturally modify their behavior to adapt to their social environment. This may involve hiding the true extent of someone's feelings and/or mood state. While altering the social presentation of emotion may be a part of everyday life, this can be especially problematic for people with depression, particularly since people are often hesitant to ask for help given the societal stigma of mental illness, which further decreases the probability of accurate diagnosis. Researchers in affective computing and social signal processing, which aim to quantify aspects of expressive behavior such as facial muscle activations and speech rate, have started looking at ways in which their communities can help mental health practitioners. This is the fundamental promise of the newly defined research field of behaviomedics, which aims to apply automatic analysis and synthesis of affective and social signals to aid objective diagnosis, monitoring, and treatment of medical conditions that alter one's affective and socially expressive behavior. Depression has been shown to correlate with the breakdown of normal social interaction, resulting in observations such as dampened facial expressive responses, avoiding eye contact, and using short sentences with flat intonation.

Unbiased, objective, continuous data capture and data analysis tools are necessary to improve current depression diagnostic practice since clinical standards for depression diagnosis are subjective, inconsistent, and imprecise. To overcome this, embodiments of the present technology simultaneously harvest visible and invisible, audible and inaudible physical cues (biomarkers) that correlate with depression, such as stress levels, head movements, activity and reaction time, psychomotor symptoms and facial expressions. This data-driven advances in contextualized affective computing and social signal processing deliver some of these objective measurements.

### Frailty

The condition being monitored or predicted by embodiments of the present technology may comprise a frailty of a user. Frailty syndrome is a common syndrome in the elderly, with a multi-factor etiology. Frailty syndrome consists of the basic characteristic of increased susceptibility to biological, physiological, and mental stressors. Impairments in multiple organ systems (such as the musculoskeletal system, the cardiovascular system, and the hematological system) cause depletion in physiological and mental reserves and decrease resistance, thereby impeding the recovery ability of the elderly as well as their ability to maintain physiological and psychosocial homeostasis. Accordingly, frailty is related to a higher prevalence of disability, falls, hospitalization, and mortality in community-dwelling older people. It is a major predictor of clinical outcomes and prognosis following any extrinsic stress, such as medical procedure, acute illness, proximal hip fracture, or hospitalization.

The understanding of frailty has evolved over the years from a basic description of dependence on others to a more dynamic model that encompasses biomedical and psychosocial aspects. It depicts a complex interplay among a person's characteristics, such as age, gender, lifestyle, socioeconomic background, morbidities, and affective, cognitive, or sensory impairments. A major requirement at present is to move from theoretical discussion on definitions of frailty to outlining practical and operational definitions that enable actual screening, assessment, and treatment of frailty, as well as differentiating frailty-related and unrelated conditions. For example, one major manifestation of frailty is functional decline, but functional decline in the elderly is part of the normal aging process and is not always an indicator of frailty. Frailty is dynamic in nature and can be viewed as a process parallel to the process of aging; and age increases the risk of frailty. The elderly population is heterogeneous in terms of chronological age and biological, psychological, and social factors, and hence, they cannot be considered a single entity. Among technology professionals who play a central role in prescription, provision, and adaptation of devices to meet the elderly's needs, there is a lack of dependable assessment tools to determine elderly behavior and needs.

As wrist-worn watch style wearables have grown in popularity, their social acceptability has grown. However, the target age group that should benefit from these advancements, the elderly, remain the least likely to use a wearable device. A significant barrier to the adoption of wearables in this user group continues to be the inconvenient form-factor, the requirement to charge devices on a daily basis, and required smartphone interface.

### Autism

The condition being monitored or predicted by embodiments of the present technology may comprise a frailty of a user. Most of the children suffering from autism have problems with learning even the basic skills required for everyday life. In general, autism is a communication disorder that requires early and continuous educational interventions on various levels like every day social interaction, communication and reasoning skills, language, understanding norms of social behavior, imagination, etc. Usually, these skills are relatively self-evident or easy to develop for other children. The devices, methods, and systems of the present technology can track and quantify individual child's specific development (sensory, communicational and interaction, motor, cognitive, social, and emotional). Additional, synchronized sensors can be tagged for context integration, environment determinants recognition of affective states and the mental workload during daily activities that elicit different emotions and states, i.e., panic, fear, frustration, anger, boredom, and sleepiness. Based on acquired affective data, interventions of the system are adapted to help and keep users in a flow state. Detection of affective states is carried out based on the physiological body signals (GSR, temperature, and embedded microsensors).

Conditions from Directed Energy Weapons (DEW)

The condition being monitored or predicted by embodiments of the present technology may be a condition from a directed energy weapon (DEW). DEW attacks may be used against corporations, governments, and/or any other individuals or organizations. For example DEW attacks may target embassies and government assets. These DEW attacks may be utilized for sensing and/or spying activities in an attempt to obtain restricted and secret information. This may be information that is being discussed, transmitted, and/or stored. For instance, listening devices may bounce energy frequencies off the window glass of an office, thus allowing the listening device to detect and/or record a private conversation occurring inside the office. DEW attacks may be deployed in a jamming and/or harassment role, such as to degrade the functionality of an embassy and/or staff. These DEW attacks may degrade cell phone reception, local area networks (for example communications from a computer to a printer), and/or any other secure forms of communication. DEW attacks may use electromagnetic frequencies in order to injure personnel and/or destroy specific forms of equipment, such as military equipment.

DEW technologies are well suited for clandestine use because some forms of DEW attacks can bypass physical walls and/or structures. The DEW attacks may directly injure targeted individuals while leaving little or no physical evidence of the attack. For example a DEW attack might not leave the conventional forensic evidence that a firearm or explosive would leave behind. DEW biophysical biomechanics relates DEW forces to disruption of anatomical regions of the human body. DEWs, including relatively high powered DEWs, create beams of energies over a broad spectrum of infrasound, radio, sonic, microwave, and/or magnetic frequencies (in narrow and/or wide-band) that may cause a range of temporary and/or permanent health effects. The DEWs may have various different parameters, depending on the type and/or configuration of the DEW, including the wavelength/frequency of the energy, mode of transmission (continuous, very rapidly pulsed, and/or slowly-to-moderately pulsed), and human health impact.

DEW signature asymptomatic and symptomatic illnesses may be impossible for a human to fake. A central tenet of DEW toxicodynamics is that there exists a relationship between DEW exposure and toxicological effects. DEW toxicokinetic-toxicodynamic (TK-TD) models assume that DEW intensity in plasma is in equilibrium and proportional with the effect site (biophase) intensity. In its simplest form a DEW Exposure Intensity versus effect plot presumes a direct relationship, where DEW effect is directly proportional to DEW Intensity at the active site and this relationship is independent of time. However, DEW physiological effects may i) manifest hours, days and/or weeks after DEW exposure. In other words, asymptomatic and/or symptomatic physiological effects may occur after the target is exposed to the DEW attack. This is termed hysteresis. When hysteresis occurs it may provide insight into the type and timing of DEW exposure from the complexity of toxic action and disposition that is encountered. Hysteresis loops may indicate that the relationship between DEW exposure intensity and the effect being measured is not a direct relationship, but may have an inherent time delay and/or disequilibrium. This delay and/or disequilibrium may be the result of the sequalae of cumulative DNA, cell, tissue and/or organ damage.

In this respect, embodiments of the present technology may comprise obtaining a baseline physiological data of the user, and comparing newly obtained physiological data to the baseline data. If an anomaly is observed, the environmental data at the time of the anomalous physiological data may be traced using time stamps, and the anomalous physiological data compared to the environmental data to identify a DWE. The signal characteristics of the identified DWE may then be used to monitor DWE events “live”.

### Other Conditions

The condition being monitored or predicted by embodiments of the present technology may comprise any one or more of pregnancy or breastfeeding tinnitus, chronic disease/infection of ENT region, ear deformation/ear surgery, degenerative or inflammatory diseases of the central nervous system, severe cognitive/neuropsychological impairment, severe pain syndrome or other severe organic diseases, epilepsy, (past or present) psychiatric disorders latent psychosis, neurological disorders, diabetic polyneuropathy, diabetic polyneuropathy, malignancies/cancer, cardiac insufficiency, arterial hypertension, heart attack/stroke, severe hepatic or renal insufficiency, diseases of the hemopoietic system, alcoholism, substance abuse, addictive personality disorder, medical history of severe allergic or toxic reactions, chronic treatment including with centrally acting medication (e.g. antipsychotics, antiepileptics, antidepressants, etc.), non-removable metal pieces (aneurysm clips, artificial limbs, etc.) or implanted electronic devices (pacemaker, osmotic or other implanted pumps, cochlear implants, etc.), claustrophobia, acute (respiratory) infection, physical uneasiness, aortic stenosis, mitral regurgitation. pulmonary hypertension, pulmonary arterial hypertension, systolic heart failure, cardiovascular diseases, coronary artery disease, hypertension, pulmonary, cardiovascular diseases, coronary artery disease, aortic valve stenosis, mitral valve insufficiency, vascular diseases, heart Failure, heart Diseases, coronary disease, myocardial ischemia, lung diseases, respiratory tract diseases, heart valve diseases, heart failure, congestive heart failure, congestive heart failure with preserved ejection fraction, ventricular outflow obstruction, artery stenosis, asymptomatic atherosclerosis, arteriosclerosis, arterial occlusive diseases, carotid atherosclerosis, blood volume measurement, spinal injury, carotid artery diseases, carotid artery stenosis, carotid disease, carotid stenosis, cerebrovascular disorders, cardiovascular diseases, endarterectomy, carotid endarterectomy, stent patency and lumen status, stroke, nervous system diseases, vascular diseases, animal health monitoring, animal husbandry, and companion animal health (e.g. cats, dogs, rodents, reptiles, large mammals, zoonotic disease tracking). Generally, additional conditions that may be diagnosed, monitored or treated include, but are not limited to infectious diseases, idiopathic pathological processes, bronchial disease, alveolar diseases, congestive obstructive pulmonary disease, respiratory hypersensitivity, immediate hypersensitivity, hypersensitivity, immune system disorders, connective tissue disorders, autoimmune diseases, lupus, asthma, undifferentiated connective tissue disease, tuberculosis, Lyme disease, mycobacterial infections, actinomycetal infections, MRSA infections, gram-positive bacterial infections, gram-negative bacterial infections, and fungal infections.

An exemplary application for the monitoring of plant health includes the monitoring of tree limbs for structural stability based on vibroacoustic signatures. For example, the weakening of a tree limb may be detected before the limb fails and falls to the ground, as in many cases, the weakening of the structural integrity of the limb may occur over time, and as cracks and tears to the limb tissues occur, they cause a detectible change in vibroacoustic signals generated by wind induced tree motions. In other cases, the sounds produced by wood boring insects or other pests may be detected and remediated before catastrophic failure occurs.

### Inanimate Structure and System Monitoring

The condition being monitored or predicted by embodiments of the present technology may comprise a condition of an inanimate structure. Similarly, in non-living, physical structures and heavy machinery rotation and natural wear and tear of rotating machines such as turbines, fans, pumps, and motors is combined with operating environment context (especially critical parameters such as temperature, humidity, barometric pressure, air quality, light exposure, noise, vibration, etc., are recorded to help determine the optimal operating state or even necessary maintenance times. This allows unnecessary wear to be avoided and possible faults and their causes to be detected early on. With the help of this monitoring, considerable optimization potential in terms of facility availability and effectiveness arises, bringing with it decisive advantages.

Metrics of improved performance include one or more of: verifiable reduction in downtimes, including prevention of catastrophic failures; extension of service life; decrease in maintenance cost; decrease in maintenance intervals; decrease in energy consumption.

In certain embodiments, with the help of ever-smarter sensors and more powerful communications networks and computing platforms, it is possible to create models, detect changes, and perform detailed calculations on service life.

### Filtering/Amplifying the Data

In certain embodiments, the method may comprise amplifying or filtering the detected signals by the foldable sensor device in order to improve a signal-to-noise ratio. In this respect, the foldable sensor device may comprise an amplifier connected to one or more of the sensors. An amplifier is a device that can attenuate frequency components of the detected signals that are not useful or needed in further processing of the detecting signals, while amplifying frequency components of the detected signals that are related or required in the further processing. In certain embodiments, the amplifier includes an adaptive filter is coupled to one of the sensors and that is configured to generate a first filtered signal by attenuating known noise components of the detected signals by the sensor. In certain embodiments, the amplifier includes a first amplifier that is coupled to a high pass filter and that is configured to generate a first amplified signal by amplifying components of the first filtered signal that are related to the signals detected by the sensors, and optionally a second amplifier that is coupled to the first amplifier and that is configured to generate a second amplified signal by amplifying components of the first filtered signal that are related to the analysis signals. The second amplifier may have an adaptive filter that is coupled to the second amplifier and that is configured to generate a second filtered signal by attenuating noise components of the second amplified signal. The amplifier may be useful for non-contact uses for ensuring an adequate noise-signal ratio, wherein the filter has a cutoff frequency within the range of any of about 0.001 Hz to 5 Hz, about 0.1 to 15 Hz, about 1 Hz to 20 Hz, and about 5 Hz-50 Hz.

### Other Data

In other aspects, the present technology may incorporate data related to Social determinants of Health (SDOH). Social determinants of health (SDOH) are conditions in the environment in which people are born, live, learn, work, play, worship, and age that affect health outcomes and risks, functioning, and quality-of-life. These social, economic and environmental conditions, in addition to health behaviors, impact and explain an estimated 80 percent of health outcomes in the United States. In order to quantify and serve people with complex clinical, behavioral health, and social needs, state the proposed solution is uniquely positioned to identify, quantify and help address these diverse environmental & social challenges. The factors that the devices, methods and systems of the present technology capture, measure and take into account may include, but are not limited to:

Income and social status, e.g. Social support networks, Education and literacy, Employment/working conditions, Social environments, Physical environments, Personal health practices and coping skills, Healthy child development, Biology and genetic endowment, Health services, Gender, and Culture.

Self-Directed Health Personal Health Record (sdhPHR):

The current “open IoT” infrastructure which is being created around the world is for the benefit of product developers and not the consumer. Most wearable business “open IoT” models are based upon data-driven approaches, passing data to a propriety cloud where data can be aggregated and mined with providing direct benefit to the consumer. Besides privacy concerns proprietary cloud firewalls limit wearable hardware, software, and data interoperability and the combination of devices which could allow actionable insights and tractable decision insights from a range of merged on-body and ambient sensors. The devices, methods and systems of the present technology allow for an extension of the existing taxonomy of electronic medical and health records, extending the concept to what is termed a personal health record (PHR) that is created and maintained by integrating a greater range, in terms of types of data, and the temporal granularity of the data than existing medical data systems allow for. Furthermore, the PHR allows for much greater control of personal data by the measured individual (the true data owner) than traditional medical records do.

### Data Definitions:

Electronic Medical Record (EMR): An electronic record of health-related information on an individual that can be created, gathered, managed, and consulted by authorized clinicians and staff within one health care organization.

Electronic Health Record (EHR): An electronic record of health-related information on an individual that conforms to nationally recognized interoperability standards and that can be created, managed, and consulted by authorized clinicians and staff across more than one health care organization.

Personal Health Record (PHR): An electronic record of health-related information on an individual that conforms to nationally recognized interoperability standards and that can be drawn from multiple sources while being managed, shared, and controlled by the individual.

The core feature of PHR, which distinguishes it from EMR and EHR, is that information contained within it is under the control of the measured individual and/or agent/controller. The above definition names such individuals as controllers, but leaves room for other bodies to act in the individual's interest, having a control over the access to PHR. Such agents may be expressly declared by the individual, though not in all cases. For example, agents acting on behalf of an individual include parents for their children, and later in life, children acting for parents.

The individual is distinctively a guardian/self-custodian of information stored or accessible within their PHR, who decides what volumes of information to include, how it is maintained and ordered, and who can read it or “check it out”. Standards and policy will need to determine if and how individuals can delete or modify information in a PHR that originated from an EHR and how these modifications are communicated to other providers with whom the data in the PHR are shared. Having control also means that an individual's PHR can exist independently of the entity that sponsors it—the PHR is portable. This requirement for portability excludes models in which sponsors such as health insurers or health care providers give individuals access to health-related information that is dependent on the individual remaining with that sponsor.

The long-term goal of a PHR is to be a lifelong resource of pertinent health information for an individual. Thus, it should have both the depth and breadth of information to enable individuals to become more engaged in their own healthcare as they move from being passive recipients to active participants in their personal health management. The health information in a PHR can be drawn from a broad range of possible sources. The sum of these and other inputs is a well-rounded picture comprising clinical information, administrative information, and wellness information for individuals to employ and impart to others at their discretion. Significant sources may include, but are not limited to:
Individuals—Self-generated information for personal management or information for care providers, including information about allergies, prescribed medications, eating habits, exercise objectives, the progression of an illness or recovery from it, and preferences regarding care in various circumstances.
Medical devices—Instruments, machines and implanted devices monitoring clinical indices, for immediate use as well as for historical purposes.
Wellness promoters—Entities supplying services or information to generate and maintain good health, such as proactive medicine centers, fitness centers, rehabilitation experts, and complementary/alternative medicine practitioners.
Health insurers—Information arising from claims for insurance payments, disease management programs recommending certain actions and collecting results, updated information on drugs in a formulary, and other coverage policies specific to an individual.
Public health—Government health departments, disease surveillance and immunization programs, school-based care providers and social workers, and nongovernmental organizations engaged in health and wellness.
Research institutions—Information about opportunities to engage in clinical trials and studies, and recently published results of interest to the individual.

The overarching theme of the present technology sdhPHR involves a patient centric, self-directed actionable insight tool that is controlled for the most part, by the individual. Their data is immediately available electronically, and can be linked to other systems, either in a “pull-push” or “push-pull” method. The present technology sdhPHR is intended to provide functionality to help an individual maintain a longitudinal view of his or her health history, and may be comprised of information from a plethora of sources—i.e., from providers and health plans, as well as from the individual.

Data collected by the system is administrative and/or clinical, and the tool may provide access to a wealth of forms (advance directives) and advice (diet, exercise, disease management). The present technology sdhPHR helps the individual collect behavioral health, public health, patient entered, and patient accessed data (including medical monitoring devices), medication information, care management plans and the like, and could be connected to providers, laboratories, pharmacies, nursing homes, hospitals and other institutions and clinical resources. At its core, the present technology sdhPHR provide the ability for the individual to capture and maintain demographic, insurance coverage, and provider information. It also provides the ability to capture health history in the form of a health summary, problems, conditions, symptoms, allergies, medications, laboratory and other test results, immunizations and encounters. Additionally, personal care planning features such as advance directives and care plans be available. The system must be secure and have appropriate identity and access management capabilities, and use standard nomenclature, coding and data exchange standards for consistency and interoperability. Optional features include record keeping of secure messaging, graphing for test results, patient education, guideline-based reminders, appointment scheduling and reminders, drug-drug interactions, formulary management, health care cost comparisons, document storage and clinical trial eligibility. The effective use of a sdhPHR is a key point for improving healthcare in terms of self-management, patient-provider communication and quality outcomes.

The present technology sdhPHR provides all necessary functionalities to assist the individual in maintaining a continuous insight into his/her medical history, including info coming from a number of sources. It assists an individual in collecting vital physiological data (e.g. from medical monitoring devices), health info, care management plans and alike, potentially connecting also to providers, labs, pharmacies, nursing homes, clinics and similar organizations and medical resources. The present technology sdhPHR encompasses the whole health history, including health issues, conditions, symptoms, allergies, medicines, lab test results, immunizations and visits. Considering the sensitivity of data on record, such platforms is secure and employs sufficient access management, authentication and authorization mechanisms.

There are two distinct dimensions offered by the present technology sdhPHR. Current solutions offer very attractive web interfaces for the patients to edit and store their personal record of health-related information. However, they completely lack any functionality related to laying out and maintenance of a rehabilitation plan. Typically, this need is covered by special purpose software solutions that are entirely clinicians-oriented and do not actively put the patients in the loop (i.e. not Personal Health Systems). In contrast, the present technology approach is patient-centric: the intention of the project is to build sdhPHR that facilitates personal care planning. This does not mean that the clinicians are made subordinates in the rehabilitation process: the proposed sdhPHR is a self-directed telemedicine enabler driven by the patient and includes healthcare workers, if granted such a right by the patients.

The present technology sdhPHR encapsulates and advances CDA/CCR/CCD/CCDA open ICT (Information and Communication Technologies) interoperability with a focus on wearable devices, self-direction, actionable insights and decision insights tracking and support.

The present technology sdhPHR offers online service for storing and maintaining various types of health-related information. The present technology sdhPHR platform has capabilities of a typical search engine, permitting users to scan specifically for medical information. Patients may store their personal medical records as well as the prescription history, manage their records, upload medical data from such devices as blood pressure monitors, glucose meters, weights, thermometers and then process this and manage this data. The information can be then shared with other types of medical and health management WEB portals and/or immediately with their physicians or general practitioners. The present technology sdhPHR includes also a desktop client allowing medical information to be received from various types of personal physiological measuring devices to be then sent to the present technology sdhPHR.

The functions are supported by the present technology sdhPHR system enable individuals to manage information about his or her healthcare. They provide direction as to the individual's ability to interact with a Personal Health Record in such a way to individualize the record and maintain a current and accurate record of his or her healthcare activities. They include activities such as managing wellness, prevention and encounters. Such functions are designed to encourage and allow an individual to participate actively in his/her healthcare and better access the resources that allow for self-education and monitoring. The principal users of these functions are expected to be individuals referenced as account holders; the patient or subject of care and healthcare providers will have access to certain functions to view, update or make corrections to their Personal Health Record. The Account Holder will receive appropriate decision support, as well as support from the present technology sdhPHR to enable effective electronic communication between providers, and between the provider and the account holder or account holder's designated representative. The sdhPHR secures controlled access to data via custom authentication ad authorization mechanisms, thus guaranteeing that all data that may be circulated around, could neither be traced back to nor be used to identify the person from whom such data had been obtained from.

### System Requirements

In certain embodiments, the system is configured to provide for wireless and/or wired connection between the processor (e.g. the smartphone/personal computer).

Environmental requirements for temperature and humidity, and fluid ingress are consistent with long term wearable devices (e.g. IP55, IP56, IP57, IP58, IP59, IP65, IP66, IP67, IP68, or IP69K to allow for effective disinfection procedures).

Embodiments of the present system may be configured to shut off or enter stand-by automatically 1, 2, 3, 4, 5, 6, 7, 8, 9 or 10 minutes after the last active operation, or after the last button is pressed.

### Sub-System Requirements

In some aspects, real-time signal acquisition, amplification, filtering, digitization, and wireless transmission are accomplished by the following integrated sub-systems:
a. Physical (autonomic system) functional state sensing subsystem using any one or more of the following sensors: Vibroacoustic sensor (Sensor in audible range, Infrasound sensor, Ultrasonic sensor); MEMS Microphone; Clinical Grade Human Body Temperature Sensor; Non-contact IR body temperature sensor; 9-Axis IMU; Ambient Light Sensor; Ambient Humidity/Pressure/Temperature; vO2, vCO2, respiratory quotient (RQ), and energy expenditure (EE), B-2 & B-52 sorties: ketones and hydration, CO2 excretion, tidal capnography; SaO2, PaO2 sensor suite; Integrated Biopotential and Bioimpedance AFE; Low-Power, Integrated In-Ear Heart-Rate Monitor; Finger Heart Rate and Pulse Oximeter Smart Sensor; Thermistor on flex foil (temperature sensor); Optical Pulse Oximeter and Heart-Rate Sensor; Health Sensor (hSensor) platform supporting the measurement of motion, precision skin temperature, bio-potential measurements (including ECG, EMG, and EEG) and reflective PPG (including pulse oximetry and heart rate); GSR; Ballistocardiography (BCG) and seismocardiography (SCG) sensors; DC-potential electromagneticcardiogram (EMCG) sensor; Ultra-Wide-Band sensor breathing analyzer; Electrochemical Sensor AFE.
b. Cognitive (central nervous system) functional state sensing subsystem comprising for example a DC-potential EEG.
c. Activity Identification and Detection sensing subsystem comprising for example one or more of: Contextual Actigraphy Sensor Tile; Ambient Light Sensor; Humidity/Pressure/Temperature; Motion Detector IC; micro barometric altimeter; barometric pressure; altitude; 9 heel lift insert and 9-Axis IMU; Full Body MotioSuit 10×9-Axis IMU; Finger Heart Rate and Pulse Oximeter Smart Sensor, and X-Band micro-Doppler radar.
d. Proximity sensing subsystem comprising for example one or more of: Haptic TactArray, Pyroelectric Energy Meter Sensor, ToF-Based Long-Range Proximity and Distance Sensor AFE, Tag-it HF-I Pro, Grid-EYE, Time-of-Flight Proximity sensor (per one or more of: Radar, Light, Terahertz, Ultrasound, mmWave); Humidity/Pressure/Temperature/X-Band micro-Doppler radar; Micro Power PIR Motion Detector IC.
e. Spatio-temporal sensing subsystem comprising for example one or more of: DigiTact Pressure Sensor; Gas sensor measuring relative humidity, barometric pressure, ambient temperature and gas (VOC); Omni/multi-directional MEMS stereo digital microphone; gyroscope for image stabilization and closed control loops; 6-axis ultra-low-power smart sensor System-in-Package; Environmental and social determinants of health sensing subsystem; and Contextual Actigraphy Sensor Tile (CAST).
f. Microcontroller unit subsystem.

In some variations, the present technology comprises an analog front end (AFE). The AFE is an integrated signal conditioning module to extract, amplify (50-100 dB gain), and filter (bandwidth 0.01 Hz-100 MHz), required to maintain high signal-to-noise ratio (SNR), high common mode rejection, and minimal baseline drift and saturation problems. The module includes coupling detection, single supply operation, adjustable gain control, adjustable low pass filter(s) (LPF), adjustable high pass filter(s) (HPF), and an integrated right-leg drive (RLD). A 50-60 Hz center frequency Wien bridge notch filter is used to remove the 50-60 Hz line frequency.

In certain variations, the present technology may comprise STMicro Cortex family processors. In other variations, the processor may be any suitable “system on a chip”, microcontrollers, or central processor units. These processors paired with a powerful dual mode BLE/BT-Classic module and an external 32, 24, 20, or 16 bit ADC, allowing acquisition of multi-parameter signals at up to 96 kHz sampling rate with the resolution of 1 microvolt (1V range). In other variations, other suitable low-power near and far field communication hardware may be used, (e.g. WiFi, photoelectric, or acoustic.)

The data harvest subsystem may be powered by a lithium (Li)-ion battery that is connected directly through a charge controller. The power management sub-module (PMM) consists of buck-boost converters (to 3.3 V and 5 V) and universal serial bus (USB-C) charger in one. The converter is based on the TPS61200 from Texas Instrumentation (TI) and has a solder or mechanical switch jumper selectable at 5 V and 3.3 V outputs and an under-voltage protection of 2.6 V. The module can be charged by a mobile charger using an on-board USB-C or any other suitable connector, or, a wireless charging system. The PMM is configured to provide 3.3 V output to the MCU, the AFE module, and the notch filter. However, any other suitable power supply may be used as long as it can supply appropriate voltages and currents to the systems and subsystems which depend on it.

The devices, methods and systems of the present technology may, in general, be powered by a variety of power supply systems. The potential power supplies for the devices, methods and systems of the present technology may comprise typical rechargeable and non-rechargeable chemical cell and battery systems such, but not limited to, lithium based systems, including, but not limited to lithium polymer, lithium phosphate, solid state lithium, lithium iron phosphate, lithium manganese oxide, lithium cobalt oxide, lithium titanate, lithium nickel cobalt aluminum oxide or lithium nickel manganese cobalt oxide. Other exemplary cell or battery types that may be used include, but are not limited to zinc-air, carbon-zinc, alkaline, or silver oxide. In some embodiments, ambient energy harvesting may be employed either as the sole power source, or as a power supply to charge the internal battery. In some embodiments, super capacitors may be used alone, or in combination with ambient energy harvesting and/or chemical cell and battery systems.

Exemplary ambient energy harvesting systems may include, but are not limited to fluid flow, photovoltaic, ambient radiation, piezoelectric, pyroelectric, thermoelectric, electrostatic, magnetic induction, metamaterial, atmospheric pressure change and other mechanical motion energy capture such as from arm, leg, plant stem or tree limb motion energy harvesting.

### Secure Communications Subsystem

The Secure communications subsystem may either be a separate system or integrated in the Microcontroller subsystem. e.g. the STM32L4A6VG contains the encryption hardware accelerator: AES (128/256-bit key), HASH (SHA-256). In other aspects, the subsystem may comprise any other suitable hardware encryption-decryption software and/or hardware.

A communication module may be configured to communicate sensor data, analysis data, and/or other information to an external computing device. Additionally, or alternatively, the communication module may communicate with external sources for microcontroller programming and software updates. The external computing device may be, for example, a mobile computing device (e.g., mobile telephone, tablet, smart watch), laptop, desktop, medical equipment, or other suitable computing device. The external computing device may be executing an application for presenting sensor data (and/or the results of analysis thereof) through a user interface to a user.

Additionally, or alternatively, the communication module may be configured to communicate data to one or more networked devices, such as a hub paired with the system, a server, a cloud network, etc. In some variations, the communication module may be configured to communicate information in an encrypted manner. While in some variations the communication module 2340 may be separate from the processor(s) as a separate device, in variations at least a portion of the communication module may be integrated with the processor (e.g., the processor may include encryption hardware, such as advanced encryption standard (AES) hardware accelerator (e.g., 128/256-bit key) or HASH (e.g., SHA-256)). Additional aspects of the communication scheme are described in further detail below with respect to the signal processing system.

The communication module may communicate via a wired connection (e.g., including a physical connection such as a cable with a suitable connection interface such as USB, mini-USB, etc.) and/or a wireless network (e.g., through NFC, Bluetooth, WiFi, RFID, or any type of digital network that is not connected by cables). For example, devices may directly communicate with each other in pairwise connection (1:1 relationship), or in a hub-spoke or broadcasting connection (“one to many” or 1:m relationship). As another example, the devices may communicate with each other through mesh networking connections (e.g., “many to many”, or m:m relationships), such as through Bluetooth mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), or any other suitable communication protocol. Some wireless network deployments may combine networks from multiple cellular networks (e.g., 3G, 4G, 5G) and/or use a mix of cellular, WiFi, and satellite communication.

In some variations, the communication module (e.g., used as input and function manipulation, and/or tactile feedback) may include multiple data communication streams or channels to help ensure broad spectrum data transfer (e.g., Opus 20 kHz with minimal delay codec). Such multiple data communication streams are an improvement over typical wireless data transmission codecs. For example, most wireless data transmission codecs (e.g., G.711) use a bandpass filter to only encode the optimal range of human speech, 300 Hz to 3,400 Hz (this is commonly referred to as a narrowband codec). As another example, some wireless data transmission codecs (e.g., G.722) encodes the range from 300 Hz to 7,000 Hz (this is commonly referred to as a wideband codec). However, most of the energy is concentrated below 1,000 Hz and there is virtually no audible sound above 5,000 Hz, while there is a measurable amount of energy above the 3,400 Hz cutoff of most codecs. The data throughput requirements for both G.711 and G.722 are the same because the modulation used in G.722 is a modified version of the PCM called Adaptive Differential Pulse Code Modulation (ADPCM). When this kind of complexity is added to a codec and process power remains constant, this will add latency. As such, G.711 will introduce latency well below just one millisecond but G.722 could introduce tens of milliseconds of delay-which is an unacceptably long delay in vibroacoustics.

In some aspects, the communications between the sensor based devices and the signal processing, general computation and ML systems may comprise CODECS disclosed in the U.S. utility application Ser. No. 17/096,806 which is hereby incorporated by reference in its entirety.

The present technology may also comprise the following subsystems:
g. App and web interface subsystem;
h. Edge inference solution, Atomic local decision support;
i. Secure Cloud data streaming subsystem;
j. Secure Cloud data storage and processing subsystem, including end-to-end encryption of stored on the sdhPHR.
k. In other aspects, the subsystems may comprise any other suitable hardware encryption-decryption software and/or hardware.
l. NSA and 3rd party APIs (Application Programming Interface) and SDKs (Software Development Kit) subsystem. E.g., Parse Server architecture with number of APIs and WebHooks for different platforms available. In other aspects, the subsystem may comprise any other suitable hardware encryption-decryption software and/or hardware.

### Usability

Embodiments of the device may be provided with one or more buttons, or other types of actuators. In certain embodiments, the number of buttons is minimized in order to maximize security, safety and hygiene. Basic controls (non-configuration) may allow for one handed operation, left or right. Basic controls can be intuitive. Basic controls including the ability to interact with the present technology solutions may be based on the orientation of the device. The user can access orientation assignments via a (drop-down menu) key assignment screen. User can reconfigure the controls based on own personal preferences. The system will include default key assignments for the platforms, which can be reset by the user of the system. The number of key presses shall be minimized without convoluting the interface with complicated button sequences. There is obviously a trade-off between number of buttons and usability.

### 3D-Printed Metamaterials

FIG. 14 illustrates two views of shapes that may be comprised by 3D printed metamaterials. In certain embodiments the device is a self-transforming, shape-conforming structures solution with the ability to adopt programmed, stochastic and random shapes for specific functions. We use composite metamaterials that self-fold using stimuli-responsive mechanisms focusing on reversible shape change to enhance their practical applicability. In this embodiment we demonstrate a method for spontaneous folding of three-dimensional (3D)-printed composites with embedded electronics into the multiple band aid size and function configurations. The composite and metamaterials are printed using a multimaterial 3D-printing process. Upon peeling from the print platform, the composite self-shapes itself using the residual forces resulting from polymer swelling during the layer-by-layer fabrication process. As a specific example, electrochromic elements are printed within the composite and can be electrically controlled through its folded legs. Our shape-transformation scheme provides a route to transform planar electronics into nonplanar geometries.

In certain embodiments, the device comprises a piezoelectric material which can change shape on application of an electrical current. The piezoelectric material may encompass or otherwise house the one or more sensors.

### Materials

The electrical variation materials used for the rigid-flex board as well as the wearable device enclosure and skin attachment methods are all focused on sensing. The flexible, waterproof materials are impervious to stretching and twisting and are coupled with tiny electrodes to record and transmit information. They rely on (but are not limited to) the following material substances:
metal-oxide-semiconductor (MOS) stacks,
various polymers,
moisture absorbing materials,
carbon nanotubes (eg., single-walled carbon nanotubes (SWCNTs) and multiwall carbon nanotubes (MWCNTs)),
metallic interconnects,
elastic rubberlike polymers to form complete powered systems that sense, measure, analyze, and communicate information,
microfluidic systems,
gold nanorods,
graphene,
rubber backing to apply device on the skin without causing irritation, polyvinyl alcohol paste, expanded polytetrafluroethylene,
calcium carbonate,
biosensitive inks that rapidly and accurately change colors (i.e., sensitive to glucose, electrolytes, pH, temperature, humidity, etc.
   Details of the Methods by which these Materials are Fabricated/Details on Assembly

Methods of manufacture of the device rely on inexpensive equipment and materials in certain embodiments. 2D Screen-printing may be combined with 3D printing for the fabrication process, which can be carried out with a relatively simple and inexpensive set of tools.

Circuits and sensors may be conductive inkjet printed, conductive pen inked, or screen printed onto commercially available sensing materials listed above that are sensor papers. The biocompatible silver screen-printable ink used is commonly used for fabricating medical devices and electrodes.

For the device substrate, printable available sensing materials listed above are used as device substrate, enclosures and reusable/recyclable skin attachment solutions

One example process of fabrication is outlined below:
1. Electronics Design-Design the circuit and/or sensors (conductive layer) to be screen-printed as the conductive layer.
2. Personalization-Use an inkjet printer to print the art layer design onto the device substrate.
3. Create Mask-Cut a negative mask of the conductive layer with a vinyl cutter for screen-printing the conductive layer.
4. Attach Mask-Apply vinyl mask onto the silkscreen.
5. Silkscreen Traces-Screen-print the circuit and/or sensors using conductive silver screen-printing ink.
6. Populate Circuit-Mount electronics onto the circuit using n-axis conductive tape at appropriate locations as required needed.
7. Apply copper tape or any desired connector to power the circuit.
8. Prepare device for Application-Apply the enclosure/adhesive layer for protecting the device and multi-use attachment.
9. Package.

### Manufacturing Details

The devices, methods and systems of the present technology may comprise highly-stretchable strain gauges. The thickness and relatively high tensile modulus of polymeric wearable devices makes them durable and highly reusable, providing the ideal substrate for encapsulating complex electronics.

The devices, methods and systems of the present technology may comprise ultra-flexible sensing circuits that include radio capability and adaptive camouflage skin overlays

The devices, methods and systems of the present technology comprise materials and construction methods that render them breathable and comfortable to wear for long periods using substrates that are uniformly thin for on-skin wearable applications and thinner, more comfortable on-skin interfaces that supports additional input/output modalities.

The devices, methods and systems of the present technology may be fabricated through the creation of a flexible conductive polymeric through the mixing of conductive materials with nonconductive polymer or by injecting liquid metal into prefabricated channels. In some aspects, the conductive polymers may be modified by mixing one or more conductive materials, such as graphite, into a nonconductive polymeric

The devices, methods and systems of the present technology may comprise materials that are well electrically performing (max conductivity) and are non-toxic.

The devices, methods and systems of the present technology are visually attractive, featuring (customizable) finish patterns. In some aspects, the patterns and textures may be modified in real-time through the use of elastomeric piezoelectric materials along with the appropriate sensing and control circuitry.

### INCORPORATIONS BY REFERENCE

Additional metrics and sensors are described in U.S. Ser. No. 17/096,806 filed Nov. 12, 2020, PCT/IB2021/053919 filed May 8, 2021 and PCT/US21/46566 filed Aug. 18, 2021, the contents of which are incorporated by reference in their entirety. Use of biometric data is further described in PCT/US2021/049161 filed Sep. 3, 2021, the contents of which are incorporated by reference in their entirety. Piezoelectric solutions may also be incorporated within the present technology, such as described in PCT/US2021/059193 filed Nov. 12, 2021 filed Nov. 12, 2020, the contents of which are incorporated by reference in their entirety.

Embodiments of the present devices or system may incorporate further components such as one or more of those described or illustrated in U.S. 63/124,632 filed Dec. 11, 2020, and PCT application claiming priority from 63/124,632 and filed concurrently with the present application and bearing attorney reference 106964/067, the contents of which are incorporated by reference in their entirety.

1. A foldable sensor device comprising:
a first substrate having a first sensor;
a second substrate having a second sensor; and
a first join member connecting the first substrate and the second substrate such that the first substrate and the second substrate are foldable relative to each other to form a folded configuration having multiple layers with the first substrate stacked relative to the second substrate.

2-3. (canceled)

4. The foldable sensor device of claim 1, wherein the first sensor is configured to detect signals from a user of the foldable sensor device and the second sensor is configured to detect signals from an environment of the user, and wherein the first sensor faces away from the second sensor.

5. The foldable sensor device of claim 4, wherein the first sensor is positioned on a first surface of the first substrate and the second sensor is positioned on a second surface of the second substrate, the first surface and the second surface being co-planar when in an unfolded configuration and stacked one above each other when in a folded configuration, with the first surface facing away from the second surface.

6. The foldable sensor device of claim 4, wherein at least a portion of one or both of the first sensor and the second sensor are formed within a first body of the first substrate or a second body of the second substrate.

7-8. (canceled)

9. The foldable sensor device of claim 4, further comprising an enclosure, wherein the enclosure has a configuration which is wearable by a user against or proximate a body part of the user and which is selected from one or more of: a strap, a band aid, a patch, a watch, a bandage, an item of jewelry, a head piece, an eye piece, an ear piece, a mouth piece, a collar, an item of clothing, a belt, a support, bedding, a blanket, a pillow, a cushion, a support surface of a seat, and a head-rest.

10. The foldable sensor device of claim 9, wherein the enclosure has a configuration suitable for non-contact uses with the user.

11. The foldable sensor device of claim 4, wherein the first sensor comprises one or more of a vibroacoustic sensor, a PPG/SpO2 sensor, and an electric potential sensor.

12. The foldable sensor device of claim 11, wherein the second sensor comprises one or more of a pressure sensor, a temperature sensor, a humidity sensor, a light sensor, and an IMU.

13. (canceled)

14. The foldable sensor device of claim 4, further comprising a battery sandwiched between the first substrate and the second substrate for providing power to one or both of the first sensor and the second sensor.

15-20. (canceled)

21. The foldable sensor device of claim 14, further comprising a processor communicatively coupled to one or both of the first sensor and the second sensor, wherein the processor is configured to receive signal data from the first sensor and the second sensor, and to identify an activity of a body in contact with or in proximity to the foldable sensor device based on the received signal data.

22. The foldable sensor device of claim 21, wherein the processor is configured to trigger, based on a data collection protocol, one or both of the first sensor and the second sensor to one or more of: start collecting data, stop collecting data, start storing the collected data and stop storing the collected data.

23-24. (canceled)

25. The foldable sensor device of claim 22, wherein the data collection protocol is based on a predetermined time interval and a trigger event.

26. The foldable sensor device of claim 25, wherein the trigger event comprises one or more of an intensity of a detected activity, an intensity of a detected signal compared to a threshold intensity, and a frequency of a detected signal compared to a threshold frequency.

27. The foldable sensor device of claim 1, wherein one or both of the first substrate and the second substrate comprises at least one connector for connecting at least one additional substrate thereto.

28. The foldable sensor device of claim 27, further comprising a third substrate connected to the first substrate or to the second substrate by a second join member such that the second join member permits stacking of the first substrate, the second substrate and the third substrate.

29. The foldable sensor device of claim 28, wherein the first substrate, the second substrate and the third substrate are foldable so that the third substrate is sandwiched between the first substrate and the second substrate.

30. The foldable sensor device of claim 1, wherein the first substrate and the second substrate are faces of a three-dimensional polyhedron which has been flattened to a two-dimensional form and re-folded.

31. The foldable sensor device of claim 1, wherein a height of the foldable sensor device is less than about 15 mm high.

32. A wearable device comprising:
an enclosure which can be placed against a body part of a user;
at least one foldable sensor device, the foldable sensor device comprising:
a first substrate having a first sensor;
a second substrate having a second sensor; and
a first join member connecting the first substrate and the second substrate such that the first substrate and the second substrate are folded relative to each other to form a folded configuration having multiple substrate layers stacked relative to one another.

33. The wearable device of claim 32, wherein the enclosure comprises one or more of: a strap, a band aid, a patch, a watch, a bandage, an item of jewelry, a head piece, an eye piece, an ear piece, a mouth piece, a collar, an item of clothing, a belt, a support, bedding, a blanket, a pillow, a cushion, a support surface of a seat, and a head-rest.

34-43. (canceled)
